(19) United States
(12) Patent Application Publication
(10) Pub. No.: US 2002/0082268 A1
(43) Pub. Date: Jun. 27, 2002

(54) Therapeutic compounds for the treatment of asthma and allergy, and methods of use thereof

(76) Inventors: Yun Gao, Southborough, MA (US); Paul Rubin, Sudbury, MA (US); Nie Xiaoyi, Boxborough, MA (US); Charles M. Zepp, Hardwick, MA (US)

Correspondence Address: FOLEY, HOAG & ELIOT, LLP, PATENT GROUP, ONE POST OFFICE SQUARE, BOSTON, MA, 02109, US

(21) Appl. No.: 09/813,096

(22) Filed: Mar. 20, 2001

Related U.S. Application Data
(60) Provisional application No. 60/190,620, filed on Mar. 20, 2000.

(51) Int. Cl. C 07D 4 3/02; A61K 31/517; C07D 239/86

(52) U.S. Cl. 514/259; 544/284; 544/287

(57) ABSTRACT

The present invention relates to compounds capable of inhibiting leukotriene activity and histamine activity, and their use in treating asthma and allergic conditions such as hay fever, dermatitus, and urticaria. Inhibition of both pathways permits more effective treatment of conditions with fewer side effects than can be achieved using most available antihistamines alone.

### RELATED APPLICATIONS

This application claims the benefit of the filing date of United States Provisional Patent Application serial No. 60/190,620, filed Mar. 20, 2000.

### BACKGROUND OF THE INVENTION

Immediate hypersensitivity diseases, including asthma, hay fever, and allergic conjunctivitis are associated with a variety of unpleasant symptoms including tearing, inflammation, and difficulty breathing. Over 35 million Americans suffer from allergic disorders such as seasonal allergic rhinitis (hay fever), twice as many as those with asthma. Furthermore, about half of asthmatics also suffer from hay fever. The physiological mechanisms which mediate these disorders are similar for all hypersensitivity diseases and generally are initiated by environmental antigens. Patients suffering from the effects of hypersensitivity diseases are predisposed to react to specific external antigens. When these antigens contact certain tissues, such as ocular, nasal, or lung tissues, these tissues become sensitized and produce undesirable and frequently life-threatening symptoms. This reaction is largely mediated by histamine and leukotrienes (for reviews, see: (a) P. R. Bernstein et al., in “Burger's Medicinal Chemistry and Drug Discovery” 1997, Fifth Ed., Vol. 5, Chapter 67, p. 405; (b) M. -Q. Zhang et al., in “Burger's Medicinal Chemistry and Drug Discovery” 1997, Fifth Ed., Vol. 5, Chapter 68, p. 495; (c) C. D. W. Brooks et al., J. Med. Chem., 1996, 39, 2629; (d) R. L. Bell et al., Ann. Report Med. Chem. 1997, 32, 91).

Histamine is widely distributed in the body. It produces various complex biological actions via interaction with specific receptors in the membranes of cell surfaces. Histamine can participate in a variety of physiological and pathological processes in different systems ranging from cardiovascular and gastrointestinal to respiratory and neuroendocrine systems. Action of histamine on H1-receptors stimulates many smooth muscles to contract, such as those in the bronchi. Histamine also increases the permeability of the capillary walls so that more of the constituents of the plasma can escape into the tissue spaces, leading to an increase in the flow of lymph and its protein content and formation of edema. Histamine has also been implicated as a mediator in asthma. Therefore, histamine H1-receptor antagonists are useful therapeutic agents for many allergic disorders, e.g., allergic rhinitis, dermatosis, urticaria, etc. Although there is compelling evidence that suggests histamine has an important pathological role in asthma, the effectiveness of antihistamines in the treatment of asthma is limited. One explanation for the low efficacy of antihistamines in asthma is that the amount of histamine (ca. 10⁻³ M) released after antigen-antibody interaction in the airway is so high that the antihistamines cannot reach a sufficiently high concentration to counteract the effect. Although newer antihistamines such as astemizole, which has attenuated sedative and anticholinergic side effects, may be used in higher doses to achieve efficacy against asthma, a risk of adverse cardiac effects arises.

In addition, the involvement of leukotrienes complicates the scenario. For example, leukotriene D4 is more than 100 times more potent than histamine as a bronchoconstrictor in humans and 1000 times more potent than platelet activating factor (PAF) in asthmatics. Leukotrienes also play a major role in the late phase of allergic reactions whereas histamine is mainly responsible for the early phase reactions.

Leukotrienes (LTs, such as LTC4, LTD4, LTE4, and LTB4) are formed from arachadonic acid through the 5-lipoxygenase (5-LO) pathway. Both in animals and in humans, leukotrienes have been shown to induce many of the features of asthma, such as bronchoconstriction, mucus hypersecretion, increased vascular permeability, pulmonary inflammatory cell recruitment, and airway hyperresponsiveness. Asthmatic airway tissue is able to generate leukotrienes after exposure to inhaled antigen and during acute asthmatic attacks. Thus, specific inhibitors of 5-LO and LTs are highly promising agents as anti-inflammatory and antiallergenic drugs. Several 5-LO and LT inhibitors have been recently approved or are in clinical trials for use against asthma.

When leukotriene antagonists are tested in animal models they do, in fact, partially block the adverse symptoms associated with hypersensitivity diseases. Furthermore when an antihistamine and a leukotriene antagonist are both administered to sensitized animals, the combination results in a near complete suppression of the symptoms. Additionally, a recent study by Merck showed that patients who combined leukotriene inhibitor montelukast with antihistamine loratidine had substantially milder hay fever symptoms than patients who used loratidine alone. These results confirm the hypothesis that a combination of antihistamines and leukotrienes is more effective for treating these disorders than antihistamines alone. A compound which can effectively modulate both contributory pathways may allow lower dosing, giving rise to fewer side effects and resulting in a less expensive therapeutic regimen than available using two separate compounds to inhibit these pathways.

### SUMMARY OF THE INVENTION

One aspect of the present invention relates to agents which exhibit inhibitory activity towards both 5-LO and H1-receptors. These compounds may be used in the treatment of diseases such as asthma and allergies, including allergic rhinitis, dermatosis, and urticaria.

### DETAILED DESCRIPTION OF THE INVENTION

The compounds of the present invention may be used to treat asthma or other allergic disorders, such as allergic rhinitis, dermatosis, and urticaria, due to their dual activities towards histamine and leukotriene pathways. Such treatment may be more effective and convenient than a combination of therapies directed to the two pathways independently. Thus, in certain embodiments, a subject compound inhibits a histamine receptor, such as the Hi receptor, with an IC₅₀ of less than 1 μM, preferably less than 100 nm, even more preferably less than 10 nm. Similarly, a subject compound may inhibit leukotriene activity, e.g., by inhibiting 5-LO or a leukotriene receptor, with an IC₅₀ less than 10 μM, preferably less than 1 μM, even more preferably less than 100 nM, and still more preferably less than 10 nM. In preferred embodiments, a subject compound shows activity towards both pathways.

### Definitions

For convenience, certain terms employed in the specification, examples, and appended claims are collected here.

The term “disease state which is alleviated by treatment with an antihistamine or leukotriene inhibitor” as used herein is intended to cover all disease states which are generally acknowledged in the art to be usefully treated with antihistamines or leukotriene inhibitors in general, and those disease states which may be usefully treated by a specific antihistamine and/or leukotriene inhibitor of the invention, such as a compound of Formula I. Such disease states include, but are not limited to, asthma and allergic reactions, such as allergic rhinitis, dermatosis, and urticaria.

The term “allergic asthma” is defined as a disorder characterized by increased responsiveness of the trachea and bronchi to various stimuli which results in symptoms which include wheezing, cough, and dyspnea.

The term “dermatitis” refers to disorder caused by inflammation to the skin including endogenous and contact dermatitis such as, but not limited to: actinic dermatitis (or photodermatitis), atopic dermatitis, chemical dermatitis, cosmetic dermatitis, dermatitis aestivalis, and seborrheic dermatitis.

The term “leukotriene inhibitor” includes any agent or compound that inhibits, restrains, retards or otherwise interacts with the action or activity of leukotrienes, such as, but not limited to, 5-lipoxygenase (“5-LO”) inhibitors, 5-lipoxygenase activating protein (“FLAP”) antagonists, and leukotriene D4 (“LTD4 ”) antagonists.

The term “ED₅₀” means the dose of a drug which produces 50% of its maximum response or effect. Alternatively, the dose which produces a pre-determined response in 50% of test subjects or preparations.

The term “LD₅₀” means the dose of a drug which is lethal in 50% of test subjects.

The term “therapeutic index” refers to the therapeutic index of a drug defined as LD₅₀/ED₅₀.

The term “structure-activity relationship (SAR)” refers to the way in which altering the molecular structure of drugs alters their interaction with a receptor, enzyme, etc.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; and the term “hydroxyl” means —OH.

The terms “amine” and “amino” are art-recognized and refer to both unsubstituted and substituted amines, e.g., a moiety that can be represented by the general formula:

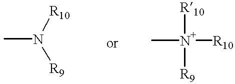

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

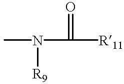

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

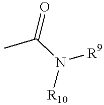

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethylthio, and the like.

The term “carbonyl” is art-recognized and includes such moieties as can be represented by the general formula:

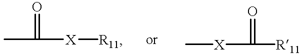

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O-(CH₂)ₘ—R₈, where m and R₈ are described above.

The term “sulfonate” is art-recognized and includes a moiety that can be represented by the general formula:

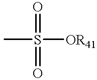

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

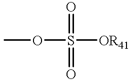

in which R₄₁ is as defined above.

The term “sulfonamido” is art recognized and includes a moiety that can be represented by the general formula:

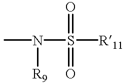

in which R₉ and R′₁₁ are as defined above.

The term “sulfamoyl” is art-recognized and includes a moiety that can be represented by the general formula:

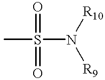

in which R₉ and R₁₀ are as defined above.

The term “sulfonyl”, as used herein, refers to a moiety that can be represented by the general formula:

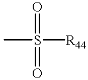

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

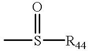

in which R₄₄ is selected from the group consisting of hydrogen, alkyl, alkenyl, alkynyl, cycloalkyl, heterocyclyl, aralkyl, or aryl.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se-(CH₂)ₘ—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as inhibitors of leukotriene activity or histamine activity), wherein one or more simple variations of substituents are made which do not adversely affect the efficacy of the compound in inhibiting the above activities. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

### Compounds of the Invention

In certain embodiments, the compounds of the present invention are represented by general structure I:

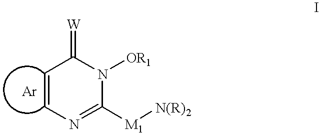

wherein

W represents O or S;

R represents independently for each occurrence H, alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted), or two instances of R taken together with the nitrogen to which they are attached may form a ring of between 3 and 8 atoms, preferably between 5 and 7 atoms, which may include 1 or 2 additional heteroatoms, and may be substituted with 1 or 2 substituents selected from alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), and —(CH₂)ₙheteroaryl (e.g., substituted or unsubstituted);

M represents, independently for each occurrence, a substituted or unsubstituted methylene group, such as —CH₂—, —CHF—, —CHOH—, or —CH(Me)—, or —C(═O)—, etc., or two instances of M taken together may form substituted or unsubstituted ethene or ethyne;

i represents an integer from 0-6, preferably from 0-3;

R₁ represents H or a substituent which may be cleaved, e.g., hydrolyzed, under physiological conditions, such as an acyl, sulfonyl, sulfinyl, phosphoryl, etc.;

n represents, independently for each occurrence, an integer from 0-10, preferably from 0-5, even more preferably from 0-3;

Ar represents a substituted or unsubstituted aryl or heteroaryl ring, e.g., fused to the depicted heterocycle; and

the stereochemical configuration at any stereocenter may be R, S, or a mixture of these configurations.

In certain embodiments, Ar represents a substituted or unsubstituted phenyl ring.

In certain embodiments, R₁ represents H.

In certain embodiments, W represents O.

In certain embodiments, M₁ includes fewer than five heavy atoms, i.e., atoms other than hydrogen. In certain embodiments, M₁ is C(R₈)₂, preferably CH₂. In certain embodiments, M represents, independently for each occurrence, a substituted or unsubstituted methylene group, such as —CH₂—, —CHF—, —CHOH—, or —CH(Me)—, or —C(═O)—, etc. In certain embodiments, M₁ is absent (i.e., i is 0) or represents lower alkyl. In certain embodiments wherein i is 1, M represents a methylene group substituted with a side chain from a naturally occurring amino acid.

In certain embodiments, N(R)₂ represents:

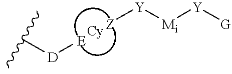

wherein

Cy represents a substituted or unsubstituted carbocyclyl, heterocyclyl, aryl, or heteroaryl ring, preferably carbocyclyl or heterocyclyl, even more preferably a six-membered ring;

D represents NR₈ or is absent;

Z represents C(YR₁), CH, C═, or N;

E represents CH or N, preferably such that at least one of Z and E is N;

Y, independently for each occurrence, represents NR₈, O, S, or is absent; and

G represents a substituted or unsubstituted heterocyclic ring; a substituted or unsubstituted aryl ring; a diarylmethyl group, optionally additionally substituted with an additional functional group, e.g., lower alkyl, YR₁, amido, etc.; or a substituted or unsubstituted polycyclic group, e.g., carbocyclic or heterocyclic, preferably including at least one aryl or heteroaryl ring, and

R₈ represents, independently for each occurrence, a functional group selected from H, alkyl, alkenyl, alkynyl, —(CH₂)ₙaralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), —(CH₂)ₙCH(aryl)₂, —(CH₂)ₙC(OH)(aryl)₂, —(CH₂)ₙpolycyclyl, and —(CH₂)ₙheteroaryl (e.g., substituted or unsubstituted), or two R₈ taken together may form a 3- to 8-membered cycloalkyl, polycyclyl, or heterocyclyl ring (e.g., substituted or unsubstituted), preferably 6- to 8-membered.

In certain embodiments, N(R)₂ represents one of:

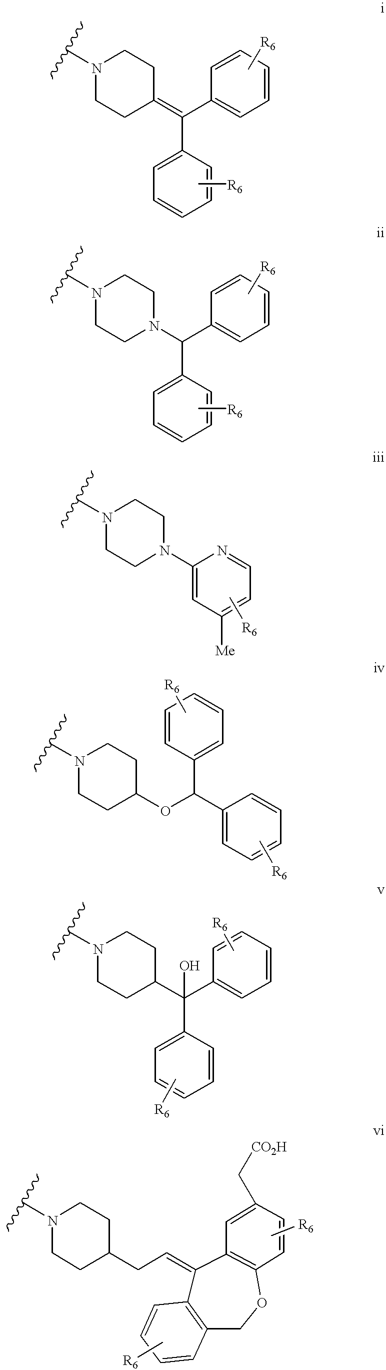

wherein R₆ represents from 0-3 substituents selected from halogen, hydroxyl, lower alkoxy, and lower alkyl, preferably from F and Cl.

In certain embodiments, a compound of the present invention is represented by general structure II:

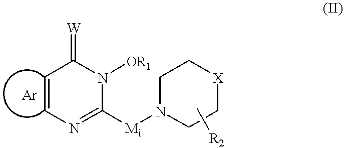

wherein

W represents O or S;

R represents independently for each occurrence H, alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted), or two instances of R taken together with the nitrogen to which they are attached may form a ring of between 3 and 8 atoms, preferably between 5 and 7 atoms, which may include 1 or 2 additional heteroatoms, and may be substituted with 1 or 2 substituents selected from alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted);

M represents, independently for each occurrence, a substituted or unsubstituted methylene group, such as —CH₂—, —CHF—, —CHOH—, or —CH(Me)—, or —C(═O)—, etc., or two instances of M taken together may form substituted or unsubstituted ethene or ethyne;

i represents an integer from 0-6, preferably from 0-3;

R₁ represents H or a substituent which may be cleaved, e.g., hydrolyzed, under physiological conditions, such as an acyl, sulfonyl, sulfinyl, phosphoryl, etc.;

n represents, independently for each occurrence, an integer from 0-10, preferably from 0-5, even more preferably from 0-3;

Ar represents a substituted or unsubstituted aryl or heteroaryl ring, e.g., fused to the depicted heterocycle;

X represents NR₈, C═C(R₈)₂, or CH(YR₈);

Y represents NR, O, S, or is absent;

R₂ represents from 0-4 substituents, preferably from 0-2 substituents, on the ring to which it is attached, and, independently for each occurrence, may represent halogen, lower alkyl, lower alkenyl, aryl, heteroaryl, carbonyl (e.g., ester, carboxyl, or formyl), thiocarbonyl (e.g., thioester, thiocarboxylate, or thioformate), ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₂ taken together with the ring to which they are attached may form a 4- to 8-membered cycloalkyl, heterocyclyl, aryl, or heteroaryl ring; and

R₈ represents, independently for each occurrence, a functional group selected from H, alkyl, alkenyl, alkynyl, —(CH₂)ₙaralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), —(CH₂)ₙCH(aryl)₂, —(CH₂)ₙC(OH)(aryl)₂, —(CH₂)ₙpolycyclyl, and —(CH₂)ₙheteroaryl (e.g., substituted or unsubstituted), or two R₈ taken together may form a 3- to 8-membered cycloalkyl, polycyclyl, or heterocyclyl ring (e.g., substituted or unsubstituted), preferably 6- to 8-membered; and

p represents an integer from 0-10, preferably from 0-5, even more preferably from 0-3.

In certain embodiments, Ar represents a substituted or unsubstituted phenyl ring.

In certain embodiments, R₁ represents H.

In certain embodiments, W represents O.

In certain embodiments, Mᵢ includes fewer than five heavy atoms, i.e., atoms other than hydrogen. In certain embodiments, Mᵢ is C(R₈)₂, preferably CH₂. In certain embodiments, M represents, independently for each occurrence, a substituted or unsubstituted methylene group, such as —CH₂—, —CHF—, —CHOH—, or —CH(Me)—, or —C(═O)—, etc. In certain embodiments, Mᵢ is absent or represents lower alkyl.

In certain embodiments, X includes a group having at least two aryl or heteroaryl rings, present either independently (e.g., in an aralkyl bearing two aryl or heteroaryl groups) or fused into a polycyclic group. In certain embodiments wherein X is C(YR₈)₂, one occurrence of R₈ is H or lower alkyl, preferably H.

In certain embodiments wherein X is C═C(R₈)₂, the two occurrences of R₈ are taken together to form a ring, preferably having one or two additional rings fused thereto.

In certain embodiments, a compound of the present invention is represented by general structure III:

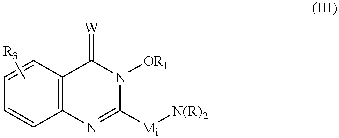

wherein

W represents O or S;

R represents independently for each occurrence H, alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted), or two instances of R taken together with the nitrogen to which they are attached may form a ring of between 3 and 8 atoms, preferably between 5 and 7 atoms, which may include 1 or 2 additional heteroatoms, and may be substituted with 1 or 2 substituents selected from alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted);

M represents, independently for each occurrence, a substituted or unsubstituted methylene group, such as —CH₂—, —CHF—, —CHOH—, or —CH(Me)—, or —C(═O)—, etc., or two instances of M taken together may form substituted or unsubstituted ethene or ethyne;

i represents an integer from 0-6, preferably from 0-3;

R₁ represents H or a substituent which may be cleaved, e.g., hydrolyzed, under physiological conditions, such as an acyl, sulfonyl, sulfinyl, phosphoryl, etc.;

n represents, independently for each occurrence, an integer from 0-10, preferably from 0-5, even more preferably from 0-3;

R₃ represents from 0-4 substituents on the ring to which it is attached, and, independently for each occurrence, may represent halogen, lower alkyl, lower alkenyl, aryl, heteroaryl, carbonyl (e.g., ester, carboxyl, or formyl), thiocarbonyl (e.g., thioester, thiocarboxylate, or thioformate), ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₃ taken together may form a 4- to 8-membered cycloalkyl, heterocyclyl, aryl, or heteroaryl ring; and

R₈ represents, independently for each occurrence, a functional group selected from H, alkyl, alkenyl, alkynyl, —(CH₂)ₙaralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted), —(CH₂)ₙCH(aryl)₂, —(CH₂)ₙC(OH)(aryl)₂, —(CH₂)ₙpolycyclyl, or two R₈ taken together may form a 3- to 8-membered cycloalkyl, polycyclyl, or heterocyclyl ring (e.g., substituted or unsubstituted), preferably 6- to 8-membered.

In certain embodiments, R₁ represents H.

In certain embodiments, W represents O.

In certain embodiments, Mᵢ includes fewer than five atoms other than hydrogen. In certain embodiments, Mᵢ is C(R₈)₂, preferably CH₂. In certain embodiments, M represents, independently for each occurrence, a substituted or unsubstituted methylene group, such as —CH₂—, —CHF—, —CHOH—, or —CH(Me)—, or —C(═O)—, etc. In certain embodiments, M₁ is absent or represents lower alkyl.

In certain embodiments, N(R)₂ represents:

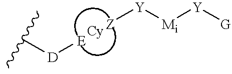

wherein

Cy represents a substituted or unsubstituted carbocyclyl, heterocyclyl, aryl, or heteroaryl ring, preferably carbocyclyl or heterocyclyl, even more preferably a six-membered ring;

D represents NR₈ or is absent;

Z represents C(YR₁), CH, C═, or N;

E represents CH or N, preferably such that at least one of Z and E is N;

Y, independently for each occurrence, represents NR₈, O, S, or is absent; and

G represents a substituted or unsubstituted heterocyclic ring; a substituted or unsubstituted aryl ring; a diarylmethyl group, optionally additionally substituted with an additional functional group, e.g., lower alkyl, YR₁, amido, etc.; or a substituted or unsubstituted polycyclic group, e.g., carbocyclic or heterocyclic, preferably including at least one aryl or heteroaryl ring.

In certain embodiments, N(R)₂ represents one of:

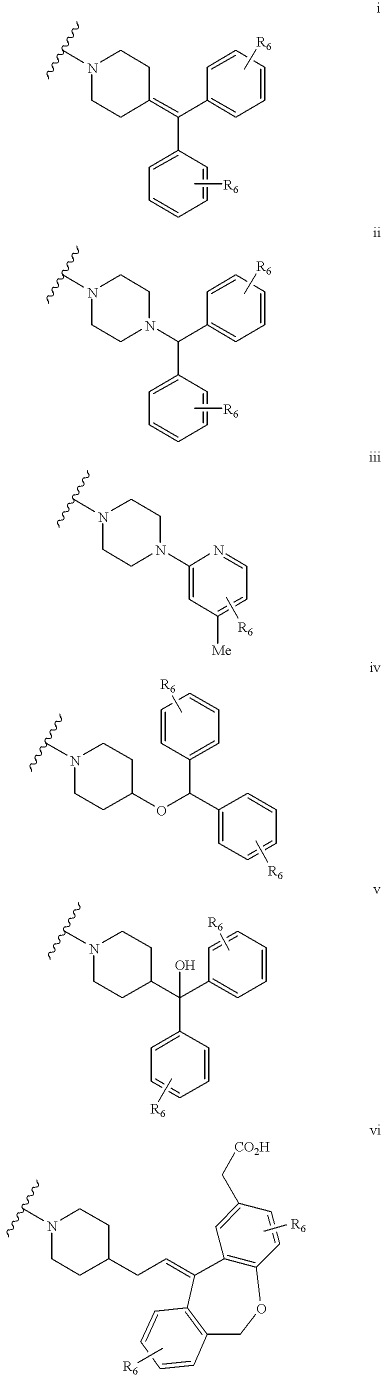

wherein R₆ represents from 0-3 substituents selected from halogen, hydroxyl, lower alkoxy, and lower alkyl, preferably from F and Cl.

In further embodiments, a compound of the present invention is represented by general structure IV:

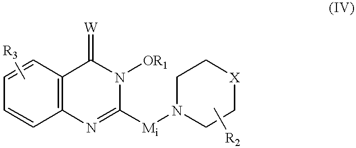

wherein

W represents O or S;

R represents independently for each occurrence H, alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted), or two instances of R taken together with the nitrogen to which they are attached may form a ring of between 3 and 8 atoms, preferably between 5 and 7 atoms, which may include 1 or 2 additional heteroatoms, and may be substituted with 1 or 2 substituents selected from alkyl, alkenyl, alkynyl, aralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), or —(CH₂)ₙheteroaryl (e.g., substituted or unsubstituted);

M represents, independently for each occurrence, a substituted or unsubstituted methylene group, such as —CH₂—, —CHF—, —CHOH—, or —CH(Me)—, or —C(═O)—, etc., or two instances of M taken together may form substituted or unsubstituted ethene or ethyne;

i represents an integer from 0-6, preferably from 0-3;

R₁ represents H or a substituent which may be cleaved, e.g., hydrolyzed, under physiological conditions, such as an acyl, sulfonyl, sulfinyl, phosphoryl, etc.;

n represents, independently for each occurrence, an integer from 0-10, preferably from 0-5, even more preferably from 0-3;

X represents NR₈, C═C(R₈)₂, or CH(YR₈);

Y represents NR, O, S, or is absent;

R₂ represents from 0-4 substituents, preferably from 0-2 substituents, on the ring to which it is attached, and, independently for each occurrence, may represent halogen, lower alkyl, lower alkenyl, aryl, heteroaryl, carbonyl (e.g., ester, carboxyl, or formyl), thiocarbonyl (e.g., thioester, thiocarboxylate, or thioformate), ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, -O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₂ taken together may form a 4- to 8-membered cycloalkyl, heterocyclyl, aryl, or heteroaryl ring;

R₃ represents from 0-4 substituents on the ring to which it is attached, and, independently for each occurrence, may represent halogen, lower alkyl, lower alkenyl, aryl, heteroaryl, carbonyl (e.g., ester, carboxyl, or formyl), thiocarbonyl (e.g., thioester, thiocarboxylate, or thioformate), ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₃ taken together may form a 4- to 8-membered cycloalkyl, heterocyclyl, aryl, or heteroaryl ring;

R₈ represents, independently for each occurrence, a functional group selected from H, alkyl, alkenyl, alkynyl, —(CH₂)ₙaralkyl, —(CH₂)ₙcycloalkyl (e.g., substituted or unsubstituted), —(CH₂)ₙheterocyclyl (e.g., substituted or unsubstituted), —(CH₂)ₙaryl (e.g., substituted or unsubstituted), —(CH₂)ₙCH(aryl)₂, —(CH₂)ₙC(OH)(aryl)₂, —(CH₂)ₙpolycyclyl, or —(CH₂)ₙheteroaryl (e.g., substituted or unsubstituted), or two R₈ taken together may form a 3- to 8-membered cycloalkyl, polycyclyl, or heterocyclyl ring (e.g., substituted or unsubstituted), preferably 6- to 8-membered; and

p represents an integer from 0-10, preferably from 0-5, even more preferably from 0-3.

In certain embodiments, R₁ represents H.

In certain embodiments, W represents O.

In certain embodiments, M₁ includes fewer than five atoms other than hydrogen. In certain embodiments, Mᵢ is C(R₈)₂, preferably CH₂. In certain embodiments, M represents, independently for each occurrence, a substituted or unsubstituted methylene group, such as —CH₂—, —CHF—, —CHOH—, —CH(Me)—, —C(═O)—, etc. In certain embodiments, Mᵢ is absent or represents lower alkyl.

In certain embodiments, X includes a group having at least two aryl or heteroaryl rings, present either independently (e.g., in an aralkyl bearing two aryl or heteroaryl groups) or fused into a polycyclic group. In certain embodiments wherein X is C(YR₈)₂, one occurrence of R₈ is H or lower alkyl, preferably H.

In certain embodiments wherein X is C═C(R₈)₂, the two occurrences of R₈ are taken together to form a ring, preferably having one or two additional rings fused thereto.

### Assays for Inhibitory Activity of Subject Compounds

One skilled in the art can measure the HI receptor affinity of proposed histamine antagonists as evaluated in rat brains or Chinese hamster ovary cells transfected with the human histamine H1 receptor gene (CHOpcDNA3H1R cells). For studies in rat brain, young male rats are sacrificed by decapitation and the brains are immediately removed. The cortici are dissected and used immediately or stored at −20° C. For studies in Chinese hamster ovary cells, confluent cells are freshly scraped from culture flasks. The tissues or cells are homogenized with a Polytron (setting no. 6 for 15 seconds) in 20 mL of 50 mM potassium sodium phosphate (pH 7.4, at 4° C.). The homogenate is centrifuged at 48,000×g for 12 minutes at 4° C. The pellet is resuspended using a Polytron (setting no. 6 for 15 seconds) in incubation buffer (50 mM potassium sodium phosphate, pH 7.4, at ambient temperature, containing 0.1% bovine serum albumin) to a concentration of 40 mg/mL and is immediately added to tubes to start the assay. The protein content of the crude membrane suspension can be determined by the method of O. H. Lowery et al., J. Biol. Chem., 193 265 (1951).

The binding assay is carried out in duplicate in 12×75 mm polypropylene tubes in 50 mM potassium sodium phosphate (pH 7.4, at ambient temperature) containing 0.1% bovine serum albumin. The radioligand, [³H]-pyrilamine, is diluted in incubation buffer to a concentration of 2 nM and added to each tube (50 μL). The test compound is diluted in incubation buffer (10⁻¹⁰ M to 10⁻⁵ M) and is added to the appropriate tubes (50 μL). The assay is started by the addition of 250 μL of well mixed tissue suspension. The final incubation volume is 0.5 mL. The assay is carried out at ambient temperature for 30 minutes. The incubation is terminated by the addition of 3.5 mL of 0.9% sodium chloride solution (4° C.) and filtration through GF/B filters that have been presoaked overnight in 0.1% polyethyleneimine, using a Brandel cell harvester. The filters are rapidly washed with two 3.5 mL portions of incubation buffer and transferred to scintillation vials. Ecolume (9 mL) is added to the vials. The vials are shaken and allowed to set for 4 hours before being counted by liquid scintillation spectrometry. Specific binding is determined as the difference between tubes containing no test compound and the tubes containing 10 μM promethazine. Total membrane bound radioactivity is generally about 5% of that added to the tubes. Specific binding is generally 75% to 90% of total binding as determined by the method of M. D. DeBacker et al., Biochem. and Biophys. Res. Commun., 197(3) 1601 (1991). The molar concentration of compound that causes 50% inhibition of ligand binding at the screening dose (10 μM) is the IC₅₀ value, and is expressed as the cumulative mean (+/−S.E.M.) for n separate experiments. For the tables presented herein, IC₅₀ values were determined by this method, but using guinea pig lung and brain tissue to assay peripheral and central H1 receptors, respectively.

Additionally, one skilled in the art can determine that the compounds of the present invention are Hi receptor antagonists in vitro by evaluating a compound's ability to inhibit histamine mediated smooth muscle contraction. Male Hartley guinea pigs, weighing 200-450 grams, are sacrificed by CO₂ asphyxiation. A piece of ileum, about 20 cm in length, is removed and cut into 2 cm pieces. Each ileum piece is placed in an organ bath at 37° C. containing Tyrode's solution and is constantly aerated with 95% O₂/5% CO₂. Tyrode's solution has the composition: sodium chloride 136.9 mM, potassium chloride 2.68 nM, calcium chloride 1.8 mM, sodium dihydrogen phosphate 0.42 mM, sodium bicarbonate 11.9 mM, and dextrose 5.55 mM. Contractions are measured with an isometric transducer (Grass FTO3C), and are recorded on a polygraph recorder and/or a computer. The ileum strips are loaded with 1.0 grams of tension and allowed to equilibrate for a minimum of 30 minutes before starting the experiments. Tissues are preincubated with vehicle or varying concentrations of test compound followed by histamine challenge.

A competitive H1 receptor antagonist produces a parallel shift of the histamine dose-response curve to the right without a depression of the maximal response. The potency of the antagonism is determined by the magnitude of the shift and is expressed as a pA₂ value which is the negative logarithm of the molar concentration of antagonist which produces a two-fold shift of the dose response curve to the right. The pA₂ value is calculated by using Schild analysis. 0. Arunlakshana and H. O. Schild, Br. J. Pharmacol Chemother. 14, 48-58 (1958). When the slope of the lines obtained by a Schild analysis are not significantly different from one (1), the compound is acting as a competitive antagonist.

One skilled in the art can determine that the compounds of the present invention mediate the immediate hypersensitivity response in vivo by evaluating the ability of the compounds to inhibit the formation of histamine (or substance P) induced wheals in guinea pigs. Animals are anesthetized with pentobarbitol (i.p.). Dorsal skin is shaved and intradermal injections of histamine (or substance P) are given in the shaved area at appropriate times after the administration of the test compounds. Doses, routes, and times of administration may vary according to experimental design. The design of such experiments is well known and appreciated in the art. Immediately after the intradermal challenges, the animal is given an intravenous injection of 1% Evan's blue dye to make the wheals visible. At an appropriate time after the challenge the animals are sacrificed by CO₂ inhalation. The skin is removed and the diameter of each wheal is measured in two perpendicular directions. The wheal response is used an the index of the edema response. The percent of inhibition of the wheal response is calculated by comparing the drug-treated group to a vehicle-treated group. Linear regression of the dose-response inhibition curve is used to determine an ED₅₀ value, expressed in mg/kg, which is the dose of compound which inhibits histamine-induced skin wheal by 50%.

The compounds of the present invention inhibit the activity of the 5-lipoxygenase enzyme. This inhibition can be demonstrated in vitro by assays using heparinized Human Whole Blood (HWB), according to the method described in Br. J. Pharmacol.: 99, pp 113-118 (1990), which determines the effect of said compounds on the metabolism of arachidonic acid. In these tests, some preferred compounds show IC₅₀ values of 0.1 to 5 μM in HWB assay, with respect to lipoxygenase activity.

Assays for inhibition of 5-lipoxygenase activity may be performed based on the following assay. The ability of a compound to reduce 5-HETE production by cell-free 5-lipoxygenase enzyme obtained from rat basophilic leukemia (RBL-1) cells is measured by a modified procedure of Cochran et al. (Biochem. Biophys. Res. Commun., 161, 1327 (1989)). RBL-1 cell-free supernatants may be prepared by a modification of the method of Jakschik et al., Prostaglandins, 25, 767 (1983). Cells (1×10⁹) may be collected by centrifugation (22° C.) and washed at 40° C. with 50 mM phosphate buffer at 5×10⁷ cells/ml. Cells were lysed on ice for 45 seconds with a Teckmar sonic disrupter. The homogenate may be centrifuged (4° C.) for 25 minutes and the supernatant (5 μg protein/μl) decanted and stored at −80° C. A 1% dimethylsulfoxide (DMSO) vehicle (DMSO in deionized water) may be utilized at various concentrations of the test compound. Duplicate reactions may be run in 35 mM buffer in the presence of 5 μM ATP, 50 μM CaCl₂ and 10 μM arachidonic acid at 37° C. The reaction may be terminated with 10 μl 0.3 citrate buffer and diluted with BGGE buffer (0.01 M phosphate, 0.1% bovine gamma globulin, pH 8.5) containing 114 μM BHT with ice bath cooling. 5-Hydroxyeicosatetraenoic acid (5-HETE) levels may be quantitated by radioimmunoassay according to standard procedures.

The leukotriene antagonistic effect may be tested in vivo on LTD4-induced bronchoconstriction in anaesthetized guinea-pigs. Intravenously, the compounds may be administered 10 minutes, orally, 24, 48 and 72 hours before the bronchoconstriction. The ED₅₀ values represent the dose inhibiting the leukotriene-induced bronchoconstriction by 50%. The ED₅₀ values may be calculated by regression analysis of 2-3 doses.

The effectiveness of subject compounds to inhibit the binding of tritiated LTB4 to guinea pig lung membranes may be determined as follows. [³H]-LTB4 (196-200 Ci/mmole) can be purchased from New England Nuclear (Boston, Mass.). All other materials can be purchased from Sigma (St. Louis, Mo.). Incubations (555 mL) may be performed in polypropylene minitubes for 45 minutes at 30° C. and containing 25 mg of guinea pig lung membrane protein (Silbaugh, et al., European Journal of Pharmacology, 223 (1992) 57-64) in a buffer containing 25 mM MOPS, 10 mM MgCl₂, 10 mM CaCl₂, pH 6.5, approximately 140 pM [³H]-LTB4, and displacing ligand or vehicle (0.1% DMSO in 1 mM sodium carbonate, final concentration) as appropriate. The binding reaction may be terminated by the addition of 1 mL ice cold wash buffer (25 mM Tris-HCl, pH 7.5) followed immediately by vacuum filtration over Whatman GF/C glass fiber filters using a Brandel (Gaithersburg, Md.) 48 place harvester.

The filters may be washed three times with 1 mL of wash buffer. Retained radioactivity may be determined by liquid scintillation counting at 50% counting efficiency using Ready Protein Plus cocktail (Beckman, Fullerton, Calif.). Nondisplaceable binding may be determined in the presence of 1 mM LTB4 and are usually less than 10% of total binding. Data may be analyzed using linear regression analysis of log-logit plots of the values between 10% and 90% of control binding to calculate IC₅₀s and slope factors (pseudo-Hill coefficients). IC₅₀ values thus obtained may be corrected for radioligand concentration (Cheng and Prusoff, Biochem. Pharmacol., 22, 3099 (1973)) to calculate Kᵢ values. pKᵢ is the mean -log Kᵢ for n experiments.

Leukotriene antagonists may be identified by observing the contractions elicited in preparations of guinea-pig ileum strips suspended in a physiological buffer by addition of pure leukotriene D4 (LTD4). When the compounds of the present invention are added to the ileum preparation before addition of LTD4 a significant inhibition of the specific LTD4-induced contraction may occur.

### Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or oral cavity; or (4) intravaginally or intravectally, for example, as a pessary, cream or foam; (5) sublingually; (6) ocularly; (7) transdermally; or (8) nasally.

The compounds according to the invention may be formulated for administration in any convenient way for use in human or veterinary medicine, by analogy with other pharmaceuticals.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and CO., San Francisco, U.S.A., 1969 or “Livestock Feeds and Feeding” O and B books, Corvallis, Oreg., U.S.A., 1977).

### Combinatorial Libraries

The subject reactions readily lend themselves to the creation of combinatorial libraries of compounds for the screening of pharmaceutical, agrochemical or other biological or medical activity or material-related qualities. A combinatorial library for the purposes of the present invention is a mixture of chemically related compounds which may be screened together for a desired property; said libraries may be in solution or covalently linked to a solid support. The preparation of many related compounds in a single reaction greatly reduces and simplifies the number of screening processes which need to be carried out. Screening for the appropriate biological, pharmaceutical, agrochemical or physical property may be done by conventional methods.

Diversity in a library can be created at a variety of different levels. For instance, the substrate aryl groups used in a combinatorial approach can be diverse in terms of the core aryl moiety, e.g., a variegation in terms of the ring structure, and/or can be varied with respect to the other substituents.

A variety of techniques are available in the art for generating combinatorial libraries of small organic molecules. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; Chen et al. (1994) JACS 116:2661: Kerr et al. (1993) JACS 115:252; PCT publications WO92/10092, WO93/09668 and WO91/07087; and the Lemer et al. PCT publication WO93/20242). Accordingly, a variety of libraries on the order of about 16 to 1,000,000 or more diversomers can be synthesized and screened for a particular activity or property.

In an exemplary embodiment, a library of substituted diversomers can be synthesized using the subject reactions adapted to the techniques described in the Still et al. PCT publication WO 94/08051, e.g., being linked to a polymer bead by a hydrolyzable or photolyzable group, e.g., located at one of the positions of substrate. According to the Still et al. technique, the library is synthesized on a set of beads, each bead including a set of tags identifying the particular diversomer on that bead. In one embodiment, which is particularly suitable for discovering enzyme inhibitors, the beads can be dispersed on the surface of a permeable membrane, and the diversomers released from the beads by lysis of the bead linker. The diversomer from each bead will diffuse across the membrane to an assay zone, where it will interact with an enzyme assay. Detailed descriptions of a number of combinatorial methodologies are provided below.

A) Direct Characterization

A growing trend in the field of combinatorial chemistry is to exploit the sensitivity of techniques such as mass spectrometry (MS), e.g., which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

B) Multipin Synthesis

The libraries of the subject method can take the multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (c.f., Bray et al. (1990) Tetrahedron Lett 31:5811-5814; Valerio et al. (1991) Anal Biochem 197:168-177; Bray et al. (1991) Tetrahedron Lett 32:6163-6166).

C) Divide-Couple-Recombine

In yet another embodiment, a variegated library of compounds can be provided on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

D) Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S.P.A. (1991) Science 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J. Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

E) Encoded Combinatorial Libraries

In yet another embodiment, the subject method utilizes a compound library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with Sequenceable Bio-oligomers

The principle of using oligonucleotides to encode combinatorial synthetic libraries was described in 1992 (13renner et al. (1992) PNAS 89:5381-5383), and an example of such a library appeared the following year (Needles et al. (1993) PNAS 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115 :2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2) Non-sequenceable Tagging: Binary Encoding

An alternative form of encoding the test compound library employs a set of non-sequencable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded.

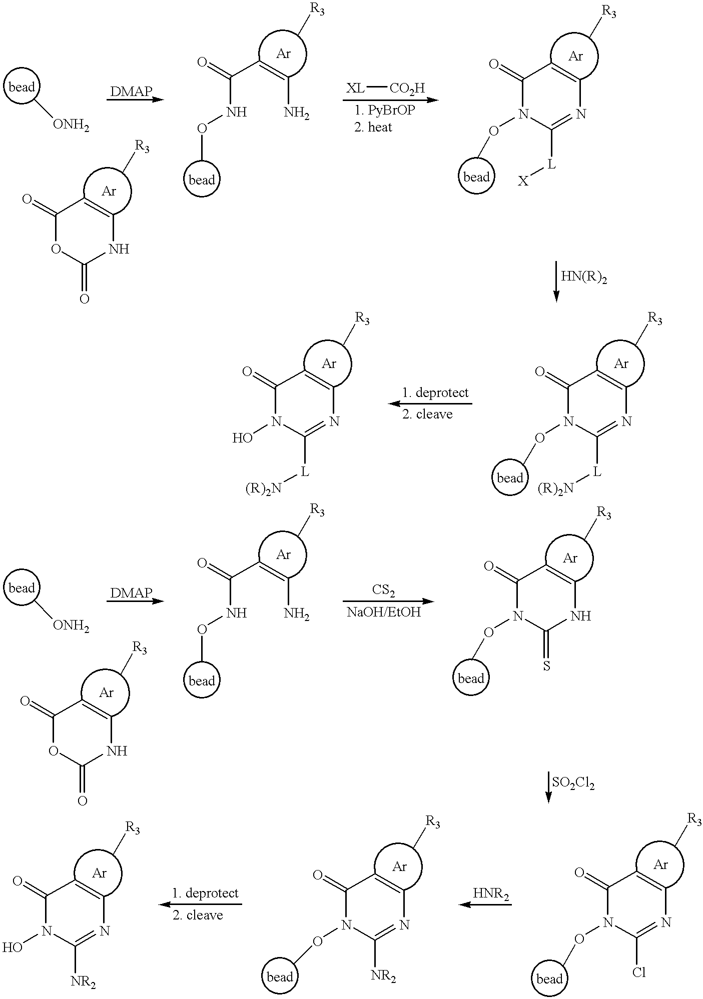

Using the above schemes, a variety of diverse compounds of the present invention may be prepared by combinatorial methods. From an initial standpoint, isatoic anhydrides having various R₃ groups may be prepared using known aromatic substitution reactions, including Stille couplings, Suzuki couplings, halogenation, etc. A variety of carboxylic acids may be used to provide diversity for L. In the above scheme, X represents a functional group on L useful for reacting with an amine. For example, X may represent a triflate or halide leaving group which can be substituted by a nucleophilic amine by an S_{N}2 mechanism. Alternatively, X may represent an aldehyde or ketone portion of L which may be coupled with an amine by reductive amination. By these methods a wide range of subject compounds may be prepared for testing in the assays described above.

### Exemplification

The invention now being generally described, it will be more readily understood by reference to the following examples, which are included merely for purposes of ilustration of certain aspects and embodiments of the present invention, and are not intended to limit the invention.

### General Procedure for LTB4 Secretion Assays

The assays were performed using human differentiated HL-60 cells using NDGA as reference compound following the literature procedure (Bennett, C. F. et al, Biochem. J., 1993, 289: 33-39). Substrate A23187 (5 uM) was incubated along with test compound in HL-60 cells at 37 C for 30 min. Reaction product LTB4 was detected by enzymeimmuno assays (EIA) using commercially available kits and a microplate reader.

Table 1 below depicts assay results for a variety of compounds of the present invention, obtained using assays as described below.

TABLE 1
Assay Results for Certain Compounds of the Invention

| | | | | Inhibition of |
|---|---|---|---|---|
| | | Binding to | Inhibition of | LTB4 |
| | | H1-receptor: | 5-LO: | secretion: |
| Entry | Structure | IC₅₀ (uM) | IC₅₀ (uM) | IC₅₀ (uM) |
| 1 | 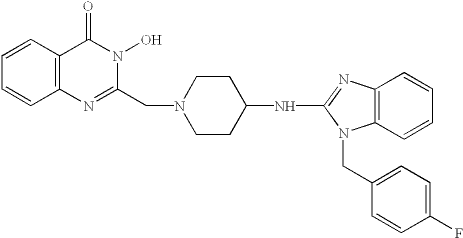 | <1 uM | <1 uM | |
| 2 | 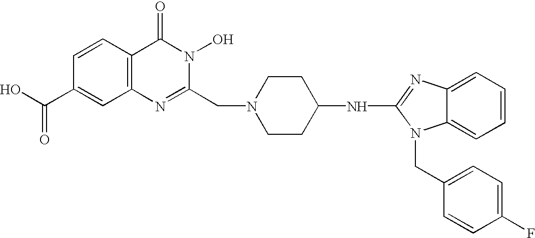 | <1 uM | >10 uM | |
| 3 | 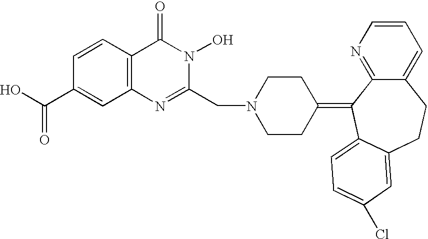 | <1 uM | <10 uM | |
| 4 | 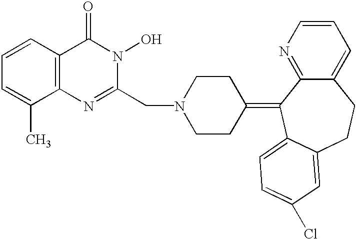 | <10 uM | <10 uM | |
| 5 | 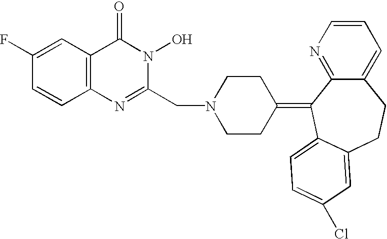 | <10 uM | <10 uM | |
| 6 | 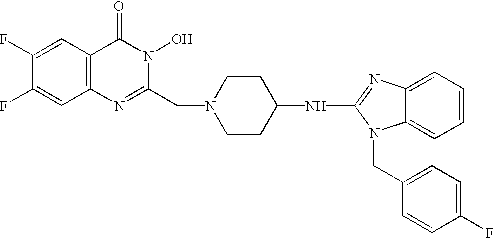 | <1 uM | <10 uM | |
| 7 | 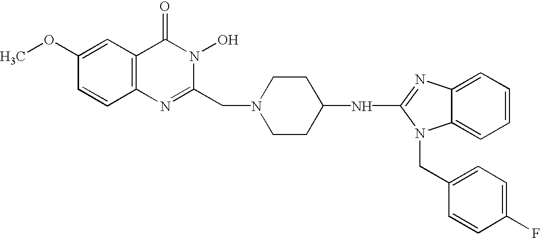 | <1 uM | <10 uM | |
| 8 | 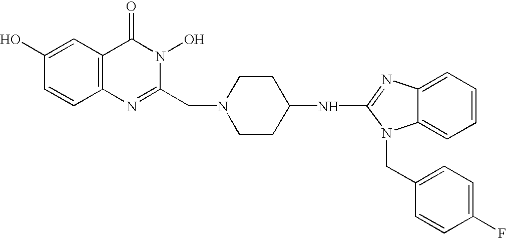 | <0.1 uM | <10 uM | |
| 9 | 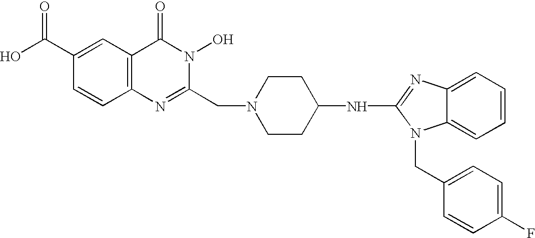 | <0.1 uM | >10 uM | |
| 10 | 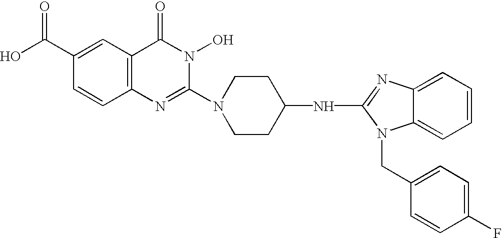 | <1 uM | >10 uM | |
| 11 | 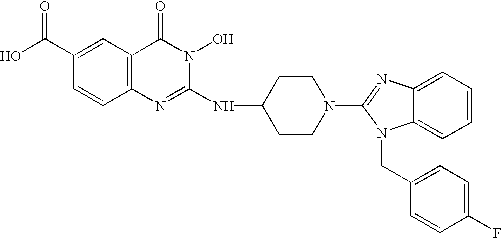 | <10 uM | >10 uM | |
| 12 | 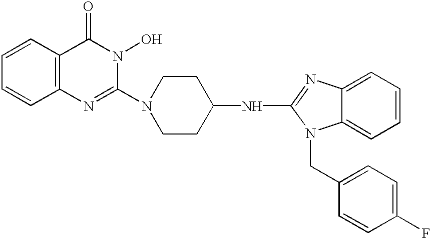 | <1 uM | <10 uM | <10 uM |
| 13 | 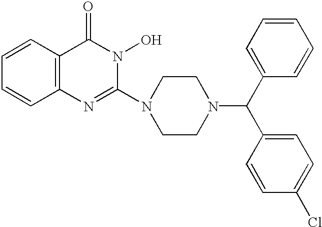 | <10 uM | | <10 uM |
| 14 | 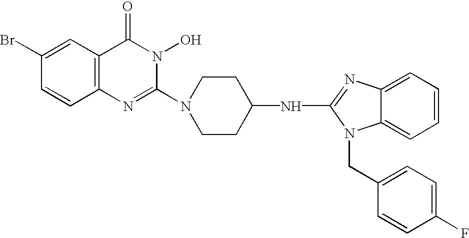 | <1 uM | <10 uM | <10 uM |
| 15 | 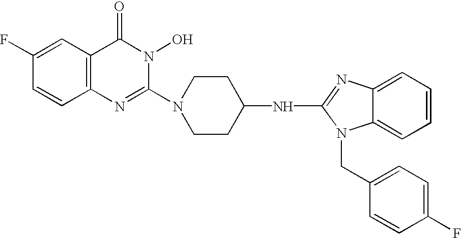 | <0.1 uM | <1 uM | <10 uM |
| 16 | 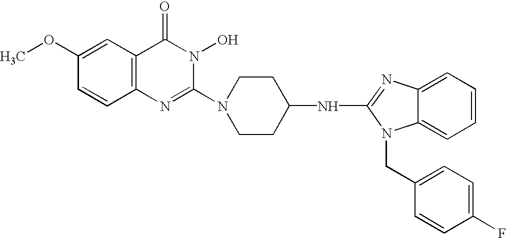 | <1 uM | <10 uM | <10 uM |
| 17 | 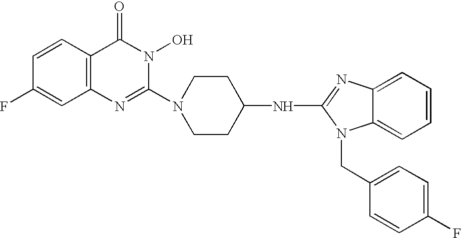 | <1 uM | | <10 uM |
| 18 | 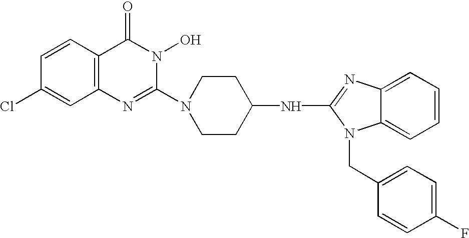 | <1 uM | | <10 uM |
| 19 | 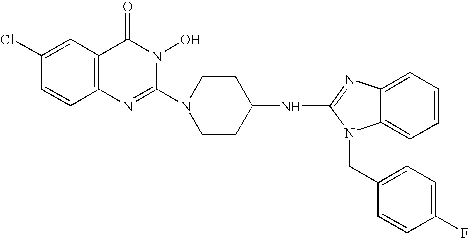 | <1 uM | <1 uM | <10 uM |
| 20 | 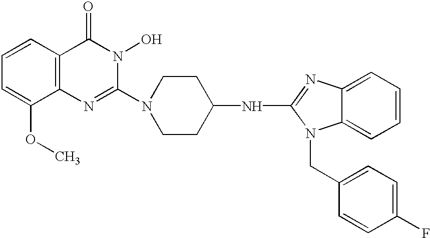 | <1 uM | | >10 uM |
| 21 | 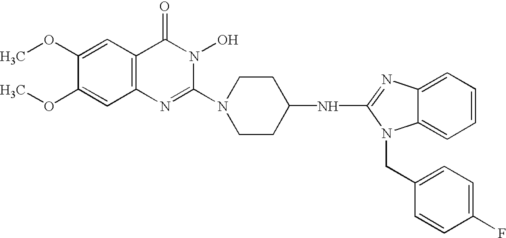 | <1 uM | | <10 uM |
| 22 | 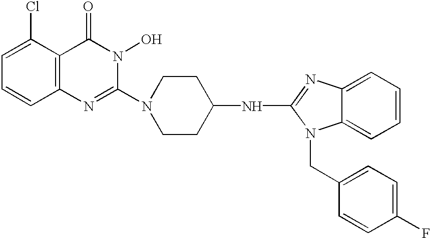 | <1 uM | | <1 uM |
| 23 | 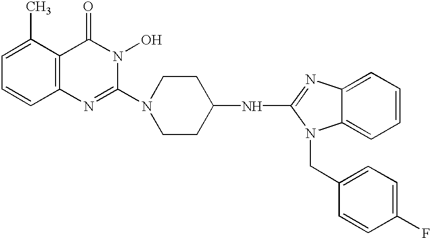 | <1 uM | | <10 uM |
| 24 | 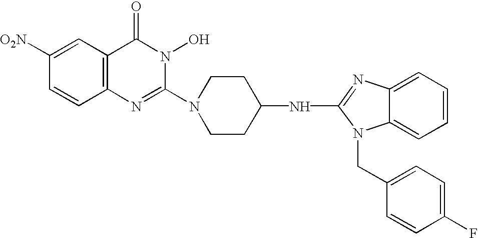 | <1 uM | | >10 uM |
| 25 | 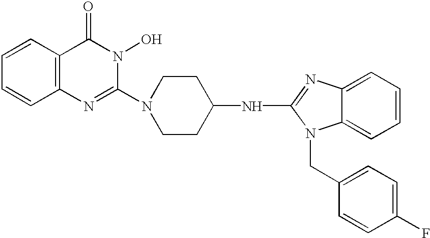 | <1 uM | | <10 uM |

I. Experimental Procedures:

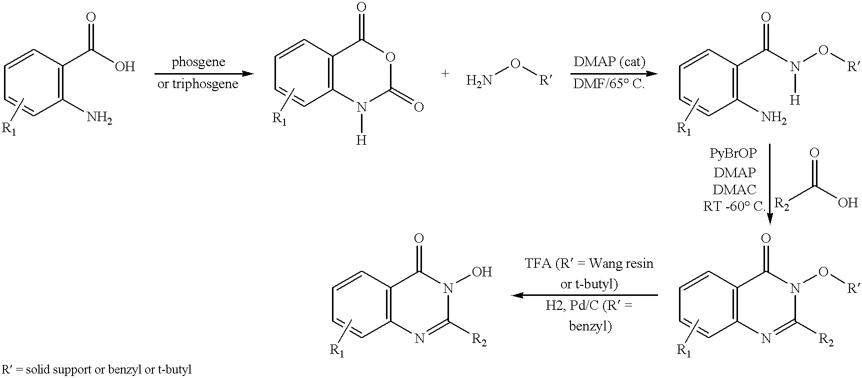

### General Procedure for the Preparation of Isatoic Anhydride (Scheme 5)

An ice-cold solution of triphosgene (3.0 g, 10 mmol, 0.4 eq) in 10 mL of anhydrous THF was added to a mixture or solution of anthranilic acid (or 2-amino-benzoic acid) (25 mmol) in 15 mL of dry THF with cooling slowly. The mixture was then stirred or shaken for 24-48 h at room temperature and cooled in a refrigerator (0° C. to −10° C.) for 12-24 h. The resulting solid was collected by filtration and washed with cold methyl t-butyl ether (MTBE) and dried to give the isatoic anhydride in 70-98% yield.

### Reaction of Isatoic anhydride with O-substituted Hydroxylamine (Scheme 5)

A mixture of isatoic anhydride (20 mmol), O-alkyldroxylamine hydrochloride (27 mmol) and K₂CO₃ (50 mmol) in 20 mL of DMF was stirred at ambient temperature or heated to 50 C. until all anhydride was consumed (24-48 h). The mixture was diluted with EtOAc and washed with water and brine. After removal of solvent, the crude product was recrystallized from EtOAc/heptane to give 2-amino-N-alkoxylbenzamide as a white solid (60-80% yd).

In this way, 2-amino-N-benzyloxybenzamides or 2-amino-N-t-butoxybenzamides are prepared.

### Preparation of O-solid-supported N-hydroxylamine on Wang Resin

Triphenyl phosphine (28.2 g, 108 mmol) was dissolved in 300 mL of dry THF. To the solution was added Wang resin (25 g, 0.86 mmol/g loading, 100-200 mesh) and N-hydroxyphthalimide (18 g, 108 mmol). The mixture was shaken until most of the N-hydroxyphthalimide was dissolved. The mixture was then cooled with ice-water and a cold solution of diethyl azodicarboxylate (17 mL, 108 mmol) in 20 mL of dry THF was added slowly with shaking and cooling. After addition, the resulting mixture was shaken at rt for 18 h. The mixture was then washed with THF, DMF, CH₃OH and CH₂Cl₂ thoroughly. The washed resin was then susppended in 300 mL of DMF and cooled with ice-water. Hydrazine hydrate (27 mL, 540 mmol) was then added. The mixture was shaken at rt for 16 h and then washed with DMF, CH₃OH and CH₂Cl₂ thoroughly and dried under vaccum to give the 0-resin supported hydroxylamine (ca. 25 g).

### Reaction of Isatoic Anhydride with Solid-supported Hydroxylamine: General Procedure (Scheme 5)

A mixture of an isatoic anhydride (3.6 mmol) and O-hydroxylamine on Wang resin (0.6 g, 1.19 mmol/g loading) and DMAP (0.18 mmol) in ca. 8-10 mL of dry DMF was stirred at 60-70° C. for 2.5 days. The mixture was then washed with DMF, CH₃OH and CH₂Cl₂ to give O-resin supported 2-amino-N-hydroxylbenzamide. A sample of the resin was cleaved with trifluoroacetic acid (TFA) in CH₂Cl₂ (1:1 v/v) to give the corresponding 2-amino-N-hydroxylbenzamide to confirm the attachment to the resin.

### General Procedure for the Synthesis of N-hydroxylquinazolinones (Scheme 5)

(A) Solid Phase Synthesis:

2-Amino-N-hydroxybenzamide on Wang resin (1.0 mmol, 0.8 mmol/g loading) was shaken with a solution of the carboxylic acid (5 mmol, 5.0 eq), PyBrOP (5 mmol, 5.0 eq) and DMAP (dimethylamino pyridine) (5 mmol, 5.0 eq) in dimethylacetamide (DMAC) (20 mL) at ambient temperature for 1 h and then at 60-65° C. for 10-12 h until the benzamide on resin is consumed. The mixture was then cooled to ambient temperature and filtered. The resin was washed thoroughly with DMF, MeOH and CH₂Cl₂ and dried. The resin is then cleaved with TFA in CH₂Cl₂ (1:1 v/v) and the filtered. The filtrate was collected and concentrated to dryness to give the desired N-hydroxylquinazolinone as brown solid. The crude product can be further purified by recrystallization or chromatography or dissolved in DMSO as stock solution for biological assay.

(B) Solution Phase Synthesis Using Carboxylic Acid Chloride:

2-Amino-N-t-butoxybenzamide (1.0 mmol ) was dissolved in DMF (4 mL). A solution of the acid chloride (1.1-1.2 mmol) in THF (4 mL) was added. The mixture was stirred at rt for 1 h and then at 65° C. for 12-16 h until no benzamide was left. The mixture was concentrated to remove THF and treated with 0.5 M NaOH solution (10 mL) at rt for 3 h with stirring. The mixture was then neutralized with dilute HC1 solution and extracted with ethyl acetate (EtOAc). The organic solution was washed with water, NaHCO₃ solution and brine. After drying, the solution was concentrated to dryness to give the crude N-t-butoxyquinazolinone. This product was treated with TFA in CH₂Cl₂ (2:1) for 3 h at rt to remove the t-butyl group. After concentration, the resulting residue was treated with NaHCO₃ solution in EtOAc. The resulting solid was filtered. The solid was then stirred with EtOAc and methyl t-butyl ether (MTBE) and filtered to give the desired pure N-hydroxyquinazolinone. If no solid is formed during NaHCO₃/EtOAc slurry, the residue was extracted with EtOAc and washed with water and NaHCO₃. After concentration, a crude product was obtained which can be recrystallized from MTBE/EtOAc or purified by chromatography on silica gel to give the desired product.

(C) Solution Phase Synthesis Using Carboxylic Acid:

A solution of 2-amino-N-t-butoxybenzamide (1.0 mmol), the carboxylic acid (1.5 mmol), PyBrOP (1.5 mmol) and DMAP (1.5 mmol) in 5 mL of DMAC was stirred at rt for 2 h and at 65° C. for 14 h. The mixture was cooled and diluted with EtOAc and washed with water, NaHCO₃ solution and NH4Cl solution. After drying and concentration, the resulting residue was treated with TFA in CH₂Cl₂ (2:1) at rt for 12 h. The mixture was concentrated and stirred with MTBE/EtOAc/H₂O to give a slurry. The slurry was filtered and the solid was collected and washed with water and MTBE and dried as the desired product. If no solid is formed during the MTBE/EtOAc/H₂O slurry, the residue was extracted with EtOAc and washed with water and NaHCO₃. After concentration, a crude product was obtained which can be recrystallized from MTBE/EtOAc or purified by chromatography on silica gel to give the desired product.

### General Procedure for the Synthesis of N-hydroxyquinazolinone Dual Inhibitors with the Methylene Bridge (Scheme 6)

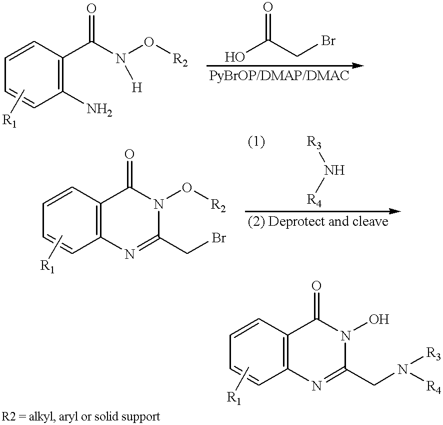

(A) Solid Phase Synthesis:

A mixture of 2-amino-N-hydroxybenzamide on Wang resin (0.04 mmol), 2-bromoacetic acid (0.2 mmmol, 5.0 eq), PyBrOP (0.2 mmol, 5.0 eq) and DMAP (0.2 mmol, 5.0 eq) in 0.6 mL of DMAC was shaken at rt for 1 h and at 60° C. for 15-20 h. The mixture was then cooled and filtered and the collected resin washed with DMF/MeOH/CH₂Cl₂ sequentially and dried. The above resin was then suspended in DMAC (0.6 mL) and treated with the amine containing the antihistamine pharmacophore (4 mmol, 10 eq) at 60-65° C. with shaking for 15-20 h. The mixture was then cooled and filtered. The collected resin was washed with washed with DMF/MeOH/CH₂Cl₂ sequentially and dried. The dried resin was then treated with TFA in CH₂Cl₂ as before to give the desired N-hydroxyquinazolinone which was dissolved in DMSO at 10 mM concentration for biological assays.

### Solution Phase Synthesis:

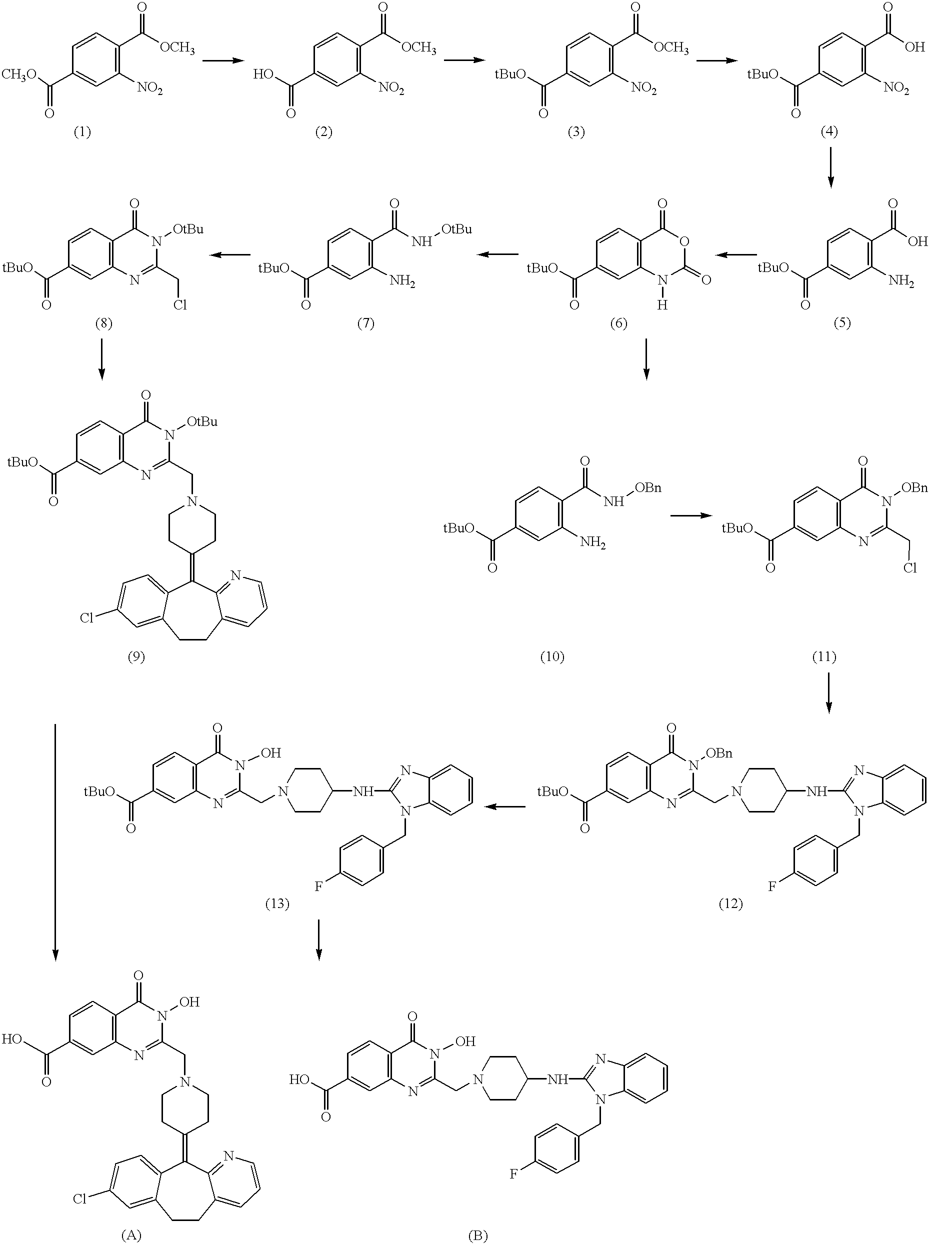

Compound 2: To a solution of dimethyl nitroterephthalate (1) (12 g, 50 mmol) in THF (100 mL) was added a solution of 1.0 N NaOH (4 g of 50% of NaOH in 50 mL of water) solution dropwise at rt. The reaction mixture was stirred at rt for 1-2 hr (or overnight). After half of THF was removed under reduced pressure, water (200 mL) was added to the residue. The aqueous phase was washed with EtOAc/MTBE (1:1) (3×200 mL) to remove unreacted starting material. The aqueous phase was acidified with diluted HCl to pH˜4, then extracted with EtOAc (2×200 mL). The extract was washed with brine and dried over anhydrous NASO₄, and then evaporated to give the crude product in good purity.

Compound 3: To a slurry of the acid (6.08 g, 27 mmol) in 25 mL of anhydrous toluene was added oxalyl chloride (67.5 mmol, 5.9 mL), and followed by two drops of DMF. The reaction mixture was stirred at rt for 1-2 hr to give a clear solution. Toluene and excess oxalyl chloride were removed under reduced pressure to give the acid chloride. To a solution of the acid chloride in CHCl₃ was added t-BuOH (67.5 mmol, 6.5 mL) and pyridine (70.2 mmol, 5.6 mL). After stirring at rt overnight, the reaction mixture was diluted with EtOAc (200 mL). Then it was washed with water (100 mL), sat'd NaHCO₃ (100 mL), brine (100 mL) and dried over NASO₄. Filtration and evaporation of the solvent under reduced pressure yielded the t-Bu-ester (7.5g), which was used in next step directly.

Compound 4: To a solution of compound 3 (7.5 g, 27 mmol) in THF (80 mL) was added a solution of 1.0 N NaOH (2.4g of 50% of NaOH in 29 mL of water) dropwise at room temperature. The reaction mixture was stirred at rt for 1-2 hr (or over night). After half of THF was removed under reduced pressure, water (200 mL) was added to the residue. The aqueous phase was washed with MTBE (100 mL) to remove unreacted starting material. The aqueous phase was acidified with diluted HCl to pH˜4, then it was extracted with EtOAc (3×150 mL). The extract was washed with water and brine, and dried over anhydrous Na₂SO₄. Filtration and evaporation afforded the crude product, which was purified by recrystallization from MTBE and hexane to give 6.2 g of compound 4 (85% yield).

Compound 5: A mixture of compound 4 (6.15 g, 23 mmol) and Pd/C (0.62 g, 10 wt. % on carbon, 50% wet) in MeOH was shaken on a Parr-shaker under 50 psi of hydrogen for about 1 hr. The mixture was then filtered through Celite to remove the catalyst. Solvent was removed under reduced pressure. Recrystallization from EtOAc and hexane gave product 5 in 86% yield (4.7 g).

Compound 6: To a solution of compound 5 (1.07 g) in THF (6 mL) was added a solution of triphosgene (1.8 mmol, 0.53 g) in THF (3 mL) slowly. The reaction mixture was shaken at rt overnight and then stored at 0° C. for 1-2 h. The resulting solid product was collected by filtration, and rinsed with MTBE and dried (1.05 g, 89% yield).

Compound 7: A mixture of compound 6 (527 mg, 2 mmol), O-t-butylhydroxylamine hydrochloride (377 mg, 3 mmol) and potassium carbonate (5 mmol, 690 mg) in DMF (4 mL) was stirred at rt overnight. The reaction was quenched with water (10 mL) and extracted with EtOAc (2×10 mL). The organic layer was washed with water (10 mL) and brine (10 mL), and dried over Na₂SO₄. Filtration and evaporation gave the crude product. Recrystallization from MTBE/hexane provided the title compound in 87% yield (530 mg).

Compound 8: To a solution of compound 7 (92.2 mg, 0.3 mmol) in DMF (0.2 mL) was added a solution of 2-chloroacetyl chloride (0.33 mmol, 0.027 mL) dropwise. After stirring at rt for 1-2 days the reaction was quenched with water, and extracted with EtOAc (2×5 mL). The organic layer was washed with sat'd NaHCO₃ (5 mL), water (5 mL) and brine (5 mL). It was dried over Na2SO₄ and evaporated to give the crude title product in 89% yield (98 mg), which was used in next step directly.

Compound 9: A mixture of compound 8 (98 mg, 0.267 mmol), descarboethoxyloratadine (DCL) (83 mg, 0.267 mmol) and DIPEA (0.4 mmol, 0.071 mL) in DMF (0.6 mL) was stirred at 60° C. for 1 hr. The reaction mixture was cooled to rt and quenched with water (5 mL) and extracted with EtOAc (2×5 mL). The organic layer was washed with water (5 mL) and brine (5 mL), and dried over Na₂SO₄. Filtration and evaporation gave 178 mg crude title product.

Compound A: A solution of compound 9 (178 mg) in TFA/CH₂Cl₂ (1: 1) was stirred at rt overnight. TFA/CH₂Cl₂ was removed under reduced pressure to give a crude product. The crude product was recrystallized from EtOAc/CH₂Cl₂ to give the title product as a TFA salt (110 mg, 64% from compound 8). The TFA salt was dissolved in HCl/EtOH (1.5 mL) and concentrated to dryness to give the crude HCl salt. The crude HCl salt was re-slurried from EtOAc to give pure title product as HCl salt.

Compound 10: A mixture of compound 6 (470 mg, 1.785 mmol), O-benzylhydroxylamine hydrochloride (455 mg, 2.85 mmol) and potassium carbonate (4.75 mmol, 660 mg) in DMF (3.8 mL) was stirred at rt overnight. The reaction was quenched with water (10 mL) and extracted with EtOAc (2×10 mL). The organic layer was washed with water (10 mL) and brine (10 mL), and dried over Na₂SO₄. Filtration and evaporation gave the crude product. Recrystallization form MTBE/hexane afforded the title compound in 69% yield (422 mg).

Compound 11: To a solution of compound 10 (297 mg, 0.867 mmol) in DMF (0.9 mL) was added dropwise a solution of 2-chloroacetyl chloride (0.954 mmol, 0.076 mL). After stirring at rt overnight the reaction was quenched with water, and extracted with EtOAc (2×10 mL). The organic layer was washed with sat'd NaHCO₃ (10 mL), water (10 mL) and brine (10 mL). It was dried over Na₂SO₄ and evaporated to give the crude title product in 93% yield (323 mg), which was used in next step directly.

Compound 12: A mixture of compound 11 (108 mg, 0.269 mmol), norastemizole (87.3 mg, 0.269 mmol) and DIPEA (0.4 mmol, 0.071 mL) in DMF (0.6 mL) was stirred at 60° C. for 1 hr. The reaction mixture was cooled to rt and quenched with water (5 mL) and extracted with EtOAc (2×5 mL). The organic layer was washed with water (5 mL) and brine (5 mL), and dried over Na₂SO₄. Filtration and evaporation gave 160 mg crude title product (87% yield).

Compound 13: A mixture of compound 12 (155 mg, 0.225 mmol) and Pd/C (30 mg, 10 wt. on carbon, 50% wet) in MeOH/EtOAc (1:1, 10 mL) was stirred under 1 atm of hydrogen overnight. The Pd/C was removed by filtering through a Celite pad. Solvent was removed under reduced pressure to give 119 mg of crude product 13 (86% yield).

Compound B: A solution of compound 13 (119 mg) in TFA/CH₂Cl₂ (1:1, 4 mL) was stirred at rt over night. TFA/CH₂Cl₂ was removed under reduced pressure to give crude product. The product was crystallized form MeOH/MTBE, and collected by filtration (as TFA salt, 126 mg, 85% yield from compound 12). The TFA salt was dissolved in HCl/EtOH (1.5 mL) and concentrated to dryness to give the crude HCl salt. The crude HCl salt was re-slurried from EtOAc to give pure title product as HCl salt.

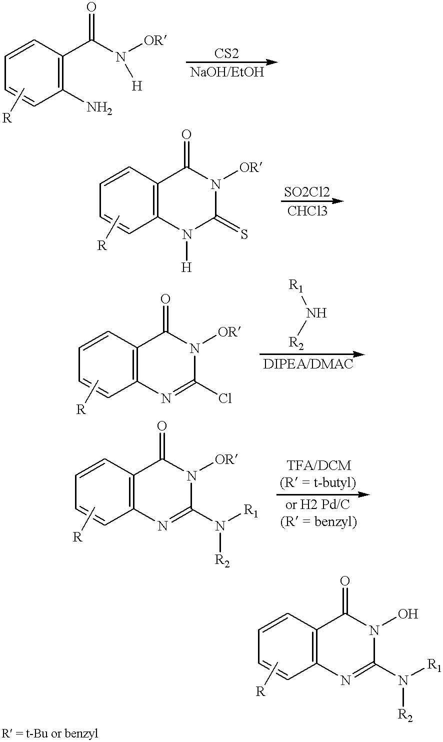

Step 1. Synthesis of 3-alkoxy-2,3-dihydro-2-thioxo-4(1H)-quinazolione (See lemura, R. et al., Chem. Pharm. Bull. 1989, 37, 2723-2726):

Sodium hydroxide (2.0 eq) and carbon disulfide (3.0 eq) were added to a solution of 2-amino-N-alkoxybenzamide (1.0 eq) in EtOH (0.5 M) and the mixture was stirred at reflux for 4-10 h. The resulting mixture was then cooled and diluted with EtOAc and washed with dilute HCl, followed by water and brie. After drying and concentration, a crude product was obtained as a yellow solid in >80% yd which was used directly for the next step or further purified by recrystallization from MTBE/EtOAc.

In this manner, 3-t-butoxy-2,3-dihydro-2-thiono-4(1H)-quinazolione and 3-benzyloxy-2,3-dihydro-2-thioxo-4(1H)-quinazolione were obtained.

Step 2. Synthesis of 2-chloro-3-alkoxy-4(3H)-quinazolinone:

Sulfuryl chloride (1.1-1.2 eq) was added to a solution of 3-alkoxy-2,3-dihydro-2-thiono-4(1H)-quinazolione (1.0 eq) in CHCl₃ (0.5 M), and the mixture was heated under reflux for 1.5-2.5 h. After cooling, the mixture was concentrated to dryness to give the crude title product which was used directly in the next step.

Step 3. Synthesis of 2-amino-3-alkoxy-4(3H)-quinazolinone:

A solution of crude 2-chloro-3-alkoxy-4(3H)-quinazolinone (1.0 eq), the amine containing the antihistamine pharmacophore (1.0 eq) and diisopropyl ethylamine (DIPEA) (1.0 eq) in DMAC (0.3 M) was heated at 85-90° C. for 3-6 h. The mixture was cooled and extracted with EtOAc and washed with water, NaHCO₃ and brine. After drying and concentration, the title product was obtained as a foamy solid which was used directly in the next step.

Step 4. Synthesis of 2-amino-3-hydroxy-4(3H)-quinazolinone (2-amino-N-hydroxyquinazolinone):

(a) From 2-amino-3-t-butoxy-4(3H)-quinazolinone:

The crude product from Step 3 was dissolved in TFA/CH₂Cl₂ (9:1 v/v) and heated at 40-60° C. for 10- 15 h to remove the t-butyl group. The solution was then concentrated to dryness and diluted with EtOAc and made basic (pH=9-10) with dilute NaOH and saturated NaHCO₃ solution. The EtOAc solution was then washed with NaHCO₃ and brine and dried. After concentration, the crude product was recrystallized from MTBE/CH₂Cl₂ and converted to the corresponding hydrochloride salt with HCl in EtOH. The HCl salt was then recrystallized from isopropyl alcohol and EtOAc to give a creamy white solid as the title product (50-55% yld).

(b) From 2-amino-3-benzyloxy-4(3H)-quinazolinone:

The crude product from Step 3 was dissolved in EtOH and hydrogenated under 1 atm of hydrogen in the presence of Pd/C catalyst until completion. The mixture was then filtered through Celite to remove the catalyst and concentrated to dryness to give the crude product which was converted to the corresponding hydrogen chloride salt as above. After recrystallization from iPrOH and EtOAc, the title product was obtained as a creamy solid.

### EXAMPLE (Scheme 7): Synthesis of Entry 12

A solution of 2-chloro-3-butoxy-4(3H)-quinazolinone (505 mg, 2.0 mmol) and 1-(p-fluorobenzyl)-2-(4-piperidylamino)benzimidazole (norastemizole) (648 mg, 2.0 mmol) and DIPEA (0.353 mL, 2.0 mmol) in 6.0 mL of DMAC was heated at 85-90° C. for 3 h. The solution was cooled and diluted with EtOAc (150 mL) and washed with water, NaHCO₃ and brine. After drying and concentration, a brown foamy solid was obtained. The solid was then treated with TFA/CH₂Cl₂ (10 ML, 9:1 v/v) at 60° C. for 12 h. The solution was concentrated and diluted with water and EtOAc. The mixture was made basic (pH=9-10) with dilute NaOH and NaHCO₃ and extracted with EtOAc (150 mL). The EtOAc solution was then washed with NaHCO₃ and brine and dried. After concentration, the crude solid obtained was recrystallized from CH₂Cl₂/MTBE. The resulting yellow solid was then treated with HCl in EtOH (1.5 M) to give the hydrogen chloride salt after removal of solvent. The crude HCl salt was recrystallized from iPrOH/EtOAc to give the title compound as a creamy white solid (620 mg, 60% yld).

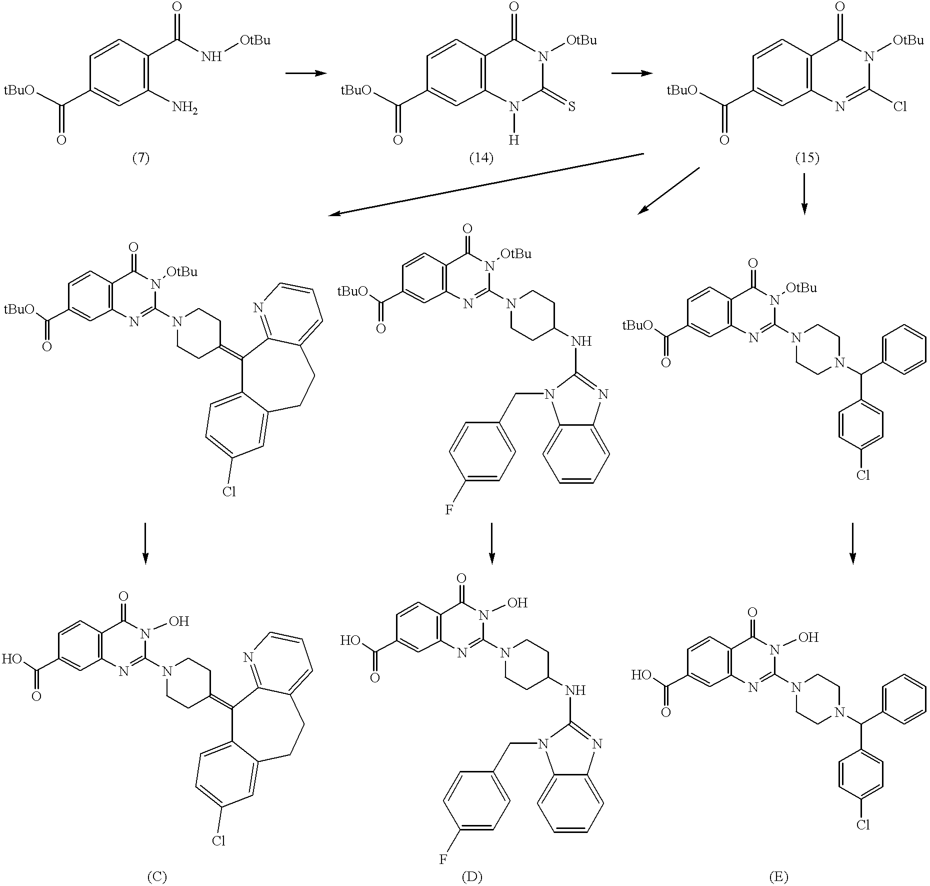

Compound 14: A mixture of compound 7 (307 mg, 1.0 mmol), carbon disulfide (5 mmol, 0.301 mL) and NaOH (1.5 mmol, 60 mg) in EtOH (1.0 ml) was stirred at 80° C. over night. The reaction mixture was cooled to room temperature and neutralized with diluted HCl. The solid was collected by filtration and recrystallized from MTBE/hexane to give compound 14 in 83% yield (290 mg).

Compound 15: To a suspension of compound 14 (290 mg, 0.827 mmol) in chloroform (2 mL) was added sulfuryl chloride (0.99 mmol, 0.080 mL). After stirring at 60-70° C. overnight the reaction was quenched with water, and extracted with EtOAc (2×10 ml). The organic layer was washed with sat'd NaHCO₃ (10 ml), water (10 ml) and brine (10 ml). White solid precipitated out from EtOAc, and collected by filtration to give title product (150 mg, 52% yield).

Compound C: A mixture of compound 15 (46 mg, 0.13 mmol), descarboethoxyloratadine (DCL) (40.4 mg, 0.13 mmol) and DIPEA (0.156 mmol, 0.028 mL) in DMAC (0.4 mL) was stirred at 90° C. overnight. The reaction mixture was cooled to room temperature and quenched with water (5 ml) and extracted with EtOAc (2×5 mL). The organic layer was washed with water (5 mL) and brine (5 mL), and dried over Na₂SO₄. Filtration and evaporation gave an oil which was treated TFA/CH₂Cl₂ (1:1, 4 mL) at room temperature for 3 hr. After concentration, the residue was re-slurried in CH₂Cl₂ to give the title product (26 mg, 32% yield from compound 15).

Compound D: Compound 15 (46 mg, 0.13 mmol) was treated with norastemizole (42 mg, 0.13 mmol) in the same fashion as for Compound C to give the title compound (21 mg, 25% yield from compound 15).

Compound E: Compound 15 (46 mg, 0.13 mmol) was treated with 1-(4-chlorobenzhydryl)-piperazine (37 mg, 0.13 mmol) as in the preparation of compound C to give the title compound (21 mg, 39% yield from compound 15).

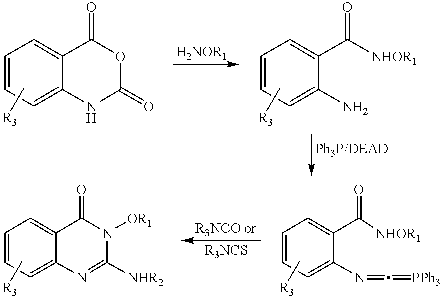

In another method, an O-substituted hydroxylamine salt may be treated with an isatoic anhydride in a polar aprotic organic solvent in the presence of a base to give the corresponding N-substituted o-aminobenzamide after usual extraction and aqueous workup. Suitable O-substituted hydroxylamines salts are O-alkylhydroxylamine salt such as commercially available O-methylhydroxylamine hydrochloride, O-allylhydroxylamine HCl, O-t-butylhydroxylamine HCl, and O-benzylhydroxylamine HCl or arylhydroxylamines/salts such as phenylhydroxylamine. Other O-substituted hydroxylamines can be made according to standard published methods. Some isatoic anhydrides are commercially available and they can be readily made by reaction of o-aminobenzoic acids (anthranilic acids) with triphosgene. Suitable polar aprotic solvents are dimethylformamide (DMF), dimethylacetamide (DMAC), dimethylsulfoxide (DMSO), dimethyl ethylene glycol (DME), tetrahydrofuran (THF). Suitable bases include K₂CO₃, Na₂CO₃, Cs₂CO₃, NaOH, KOH. The reaction temperature is in the range of 40-80° C. The N-substituted o-aminobenzamides are generally isolated and purified by recrystallization or column chromatography on silica gel.

The benzamide may then be reacted with a phosphine, such as triphenylphosphine, tritolylphosphine, trianisylphosphine, trifurylphosphine, etc. (e.g., 1.3-1.5 eq), and of an azodicarboxylate ester such as diethylazodicarboxylate (DEAD), dimethylazodicarboxylate (DMAD), di-t-butylazodicarboxylate, ethyl methylazodicarboxylate, etc. (e.g., 1.3-1.5 eq), e.g., in THF at 0° C.-rt for 12-24 h to give the corresponding iminophosphorane which is isolated and purified by recrystallization or column chromatography on silica gel. Alternatively, an activated phosphine reagent, preferably a triarylphosphine, may be prepared by combining a suitable phosphine with an elemental halogen (e.g., I₂, Cl₂, Br₂, etc.) to form a species such as Ar₃PX₂; a phosphine oxide may be combined with a reagent such as triflic anhydride, tosyl chloride, oxalyl chloride, etc., which converts the oxygen of the phosphoryl group into a leaving group (e.g., Br, Cl, I, OTs, OTf, etc.) to form a species such as Ar₃PX₂ wherein Ar represents an aryl or heteroaryl group and X represents a suitable leaving group, or any other method of preparing a phosphorous (V) reagent which can react with an aniline compound to form an iminophosphorane.

The iminophosphorane may then be treated with 1.5-2.0 eq of an isocyanate in a polar aprotic solvent such as DMF or DMAC at 70-90° C. for 20-48 h to give the 2-amino-3-alkoxy- or 3-aryloxy-4-quinazolinone after column chromatography on silica gel or recrystallization from a suitable solvent such as MeOH. Isothiocyanates can be used for the above transformation in some instances.

### Solid-phase synthesis of 2-amino-3-hydroxy-4-quinazolinones

In a preferred embodiment of the disclosure, a resin-bound O-hydroxylamine (ref: Floyd, C. D. et al, Tetrahdedron Lett. 1996, 37, 8045; Richter, L. S. et al, Tetrahedron Lett. 1997, 38, 321; Mellor, S. L. et al, Tetrahedron Lett. 1997, 38, 3311) is treated with an excess (2-5 eq) of an isatoic anhydride in the presence of dimethylaminopyridine (DMAP) (0.5-1.0 eq) in a polar aprotic solvent such as DMF, DMAC or N-methylpyrrolidinone (NMP) at 50-70° C. to give the resin-bound o-aminobenzamide. The resin is selected in such as way that it can be cleaved under mild conditions such as TFA acidic cleavage to give the free hydroxyl group. Examples of suitable resins are Wang resin, HMPB-MBHA resin, and trityl polystyrene resin.

The resin-bound O-aminobenzamide may then be treated with Ph₃P and DEAD in THF at rt according to the literature procedure (Wang, F. et al, Tetrahedron Lett. 1997, 38, 8651) to give the resin-bound aryl iminophosphorane.

The resin bound iminophosphorane may then be reacted with an excess of an isocyanate or isothiocyanate in a polar aprotic solvent such as DMF or DMAC at rt for 1-2 h followed by heating at 70-100° C. for 20-40 h to give the resin bound quinazolinone. In order to obtain the 3-hydroxyquinazolinone, the resin is treated with TFA in DCM. The resin is filtered and washed, and the filtrates are collected and concentrated to dryness under vacuum to give the desired 2-amino-3-hydroxy-4-quinazolinone.

Unlike sterically unhindered alkyl or aryl isocyanates, sterically hindered isocyanates such as t-butyl isocyanate and admantyl isocyanate and alkyl or aryl isothiocyanates may not react with resin-bound iminophosphoranes under the above reaction conditions.

The solid-phase procedure described above is summarized in the following scheme:

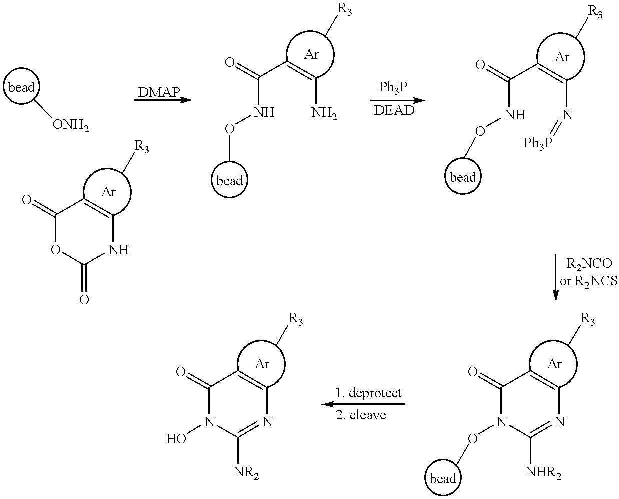

(3) Synthesis of a library of 2-amino-3-hydroxy-4-quinazolinones on solid support:

In another preferred embodiment of the disclosure, a library of quinazolinones are prepared via solid-phase reaction using split-pool method. Thus, six resin-bound substituted O-aminobenzamides were pooled into two group of three resins each. The two group of resins were then reacted with Ph₃P (6.0 eq) and DEAD (6.0 eq) in THF at rt for 24 h, respectively. The resins were then washed with THF and CH₂Cl₂ and dried. Each group of resins was then distributed into 45 vials and was treated with 45 isocyanates (10 eq) at rt for 12-20 h in DMAC in the presence of DMAP (2.0 eq) and at 70° C. for 20-24 h. The resins were then washed with DMF, MeOH and CH₂Cl₂ and dried. The desired 2-amino-3-hydroxy-4-quinazolinones were then obtained by cleavage from the resins using 50% TFA in CH₂Cl₂, followed by vacuum drying. In this way a library of 270 compounds was obtained as mixture of three compounds per vial.

(1) General Procedure for preparation of 2-amino-N-alkoxybenzamides:

A mixture of isatoic anhydride (5 mmol, 1.0 eq), O-substituted hydroxylamine hydrochloride salt (1.5 eq, 7.5 mmol), and K₂CO₃ (2.5 eq, 12.5 mmol) in DMF (5 mL) was stirred at rt for 26 h. The mixture was then quenched with water and extracted with 3×50 mL of EtOAc. The EtOAc phase was then washed with water and saturated NaCl solution and dried over Na₂SO₄. After removal of the solvent, the crude product was purified by recrystallization from EtOAc and heptane as solid (yield range 26-80%) (higher yield could be obtained if purified by column chromatography on silica gel).

(2) General Procedure for preparation of 2-iminophosphorane N-alkoxybenzamides:

2-Amino-N-alkoxybenzamide (1.0 eq, 1.0 mmol) and Ph₃P (1.3-1.5 mmol) were dissolved in dry THF (5 mL) and the solution was cooled with ice to ca. 0-5° C. DEAD (1.3-1.5 mmol) was added dropwise to the solution. The resulting mixture was stirred at rt for 15-24 h. The solution was then concentrated to dryness and the residue was recrystallized from EtOAc and heptane to give a solid product (yield range 64-97%).

2-Iminophosphorane N-benzyloxybenzamide (75% yield); ¹H NMR (CDCl₃, 300 mHz) ppm: 8.22 (br.d, 1 H), 7.1-7.6 (m, 22 H), 6.9 (t, 1H), 6.75 (t, 1H), 5.0 (s, 2H).

2-Iminophosphorane 5-chloro-N-benzyloxybenzamide (79% yield); ¹H NMR (CDCl₃, 300 mHz) ppm: 8.2 (br. S, 1 H), 7.1-7.6 (m, 22 H), 6.8 (m, 1 H), 5.0 (s, 2H).

2-Iminophosphorane 5-nitro-N-benzyloxybenzamide (81% yield); ¹H NMR (CDCl₃, 300 mHz) ppm: 9.2 (m, 1 H), 7.1-7.7 (m, 23 H), 5.0 (s, 2 H).

2-Iminophosphorane 3-methoxy-N-benzyloxybenzamide (65% yield); ¹H, NMR (CDCl₃, 300 mHz) ppm: 7.9 (m, 1H), 7.4-7.6 (m, 17H), 7.2 (br.s, 5H), 6.7 (t, 1H), 4.82 (s, 2H), 3.82 (s, 3H).

2-Iminophosphorane 5-methoxy-N-t-butoxybenzamide (64% yield); ¹H NMR (CDCl₃) ppm: 7.4-7.6 (m, 18H), 6.5 (m, 1H), 3.78 (s, 3H), 1.2 (s, 9H).

2-Iminophosphorane 5-chloro-N-t-butoxybenzamide (60% yield); ¹H NMR (CDCl₃) ppm: 8.2, (bs, 1H), 7.5-7.7 (m, 17H), 6.8 (m, 1H), 1.2 (s, 9H).

(3) General Procedure for preparation of 2-amino-3-alkoxy-4-quinazolinones:

The iminophosphorane from procedure 2 (1.0 eq) was dissolved in dimethylacetamide (DMAC) (0.5 M concentration of iminophosphorane). An isocyanate (1.5-3.0 eq) was added and the resulting solution was heated at 65-80° C. for 1-3 days. The solution was diluted with EtOAc and washed with water, NaHCO₃ and NaCl solutions and dried over Na₂SO₄. After removal of solvent, the crude product was purified by recrystallization from MeOH or column chromatography to give the pure title compound (yield range 26-97%).

2-(4′-Nitrophenylamino)-3-benzyloxy-4-quinazolinone (66% yield); ¹H NMR (DMSO-D₆) ppm: 9.8 (s, 1H), 7.3-8.2 (m, 13H), 5.3 (s, 2H).

2-(4′-Bromophenylamino)-3-benzyloxy-6-bromo-4-quinazolinone (68% yield); ¹H NMR (DMSO-D₆) ppm: 9.4 (s, 1H), 8.1 (m, 1H), 7.2-7.8 (m, 11H), 5.3 (s, 2H).

2-(Phenylamino)-3-benzyloxy-6-chloro-4-quinazolinone (64% yield); ¹H NMR (DMSO-D₆) ppm: 9.3 (s, 1H), 7.95 (s, 1H), 7.1-7.7 (m, 12H), 5.3 (s, 2H).

2-(Benzylamino)-3-benzyloxy-4-quinazolinone (26% yield), ¹H NMR (CDCl₃) ppm: 10.4 (bs, 1H), 7.7 (d, 1H), 7.1-7.6 (m, 12H), 6.95 (d, 1H), 5.25 (s, 2H) 4,6 (d, 2H ),

2-(Benzylamino)-3-benzyloxy-6-bromo-4-quinazolinone (97% yield), ¹H NMR (CDCl₃): 8.25 (m, 1H), 7.65 (m, 1H), 7.2-7.5 (m, 12H), 5.6 (m, 1H), 5.25 (s, 2H), 4.5 (d, 2H).

2-(n-Pentylamino)-3-benzyloxy-6-nitro-4-quinazolinone (63% yield), ¹H NMR (CDCl₃): 9.0 (s, 1H), 8.4 (m, 1H), 7.35-7.6 (m, 7H), 5.55 (m, 1H), 5.3 (s, 2H), 3.35 (m, 2H), 1.2-1.6 (m, 6H), 0.9 (t, 3H).

2-(Cyclohexylamino)-3-benzyloxy-6-chloro-4-quinazolinone (80% yield), ¹H NMR (CDCl₃) 8.1 (s, 1H), 7.4-7.6 (m, 6H), 7.15 (s, 1H), 5.3 (s, 2H), 5.2 (d, 1H), 3.8 (m, 1H), 1.9 (m, 2H), 1.6 (m, 4H), 1.0-1.4 (m, 4H).

2-(Phenylamino)-3-allyloxy-6-chloro-4-quinazolinone (50% yield), ¹H NMR (CDCl₃): 8.1 (s, 1H), 7.7 (dd, 2H), 7.55 (dd, 1H), 7.4 (m, 4H), 7.2 (dt, 1H), 6.1 (m, 1H), 5.5 (dd, 2H), 4.9 (d, 2H).

2-(4′-Iodophenylamino)-3-t-butoxy-6-chloro-4-quinazolinone (60% from 4-iodophenyl isothiocyanate), ¹H NMR (CDCl₃) 8.1 (d, 1H), 7.4-7.8 (m, 6H), 1.5 (s, 9H).

(4) Synthesis of resin-bound 2-amino-O-hydroxybenzamide:

O-resin bound N-hydroxylamine was prepared from Wang resin according to Floyd (Floyd, C. D., et al, Tetrahedron Lett. 1996, 37, 8045). The resin (1.0 eq, 1.0 g, 0.86 mmol) and an isatoic anhydride (5.0 eq, 4.3 mmol) and DMAP (0.3 eq, 0.25 mmol) in dry DMF (15 mL) were shaken at 60-70° C. for 1-2 days. The resin was then filtered and washed thoroughly with DMF, MeOH and CH₂Cl₂ and dried to give resin bound 2-amino-N-hydroxylbenzamide.

(5) General Procedure for synthesis of 2-iminophosphorane N-hydroxylbenzamide on resin:

The resin bound 2-amino-N-hydroxylbenzamide from procedure 4 (1.0 eq) was suspended in dry THF. Ph₃P (6.0 eq) was added and the mixture was shaken to dissolve Ph₃P and cooled with ice. Diethylaminodicarboxylate (DEAD) (5.0 eq) (neat or in THF solution) was added. The resulting suspension was shaken at rt for 18-24 h. The resin was then filtered and washed with THF and CH₂Cl₂ and dried to give 2-iminophosphorane N-hydroxylbenzamide on resin (sample was cleaved with TFA/CH₂Cl₂ and analyzed by HPLC/MS to confirm the formation of the iminophosphorane).

(6) General Procedure for synthesis of 2-amino-3-hydroxy-4-quinazolinones:

The Wang resin bound 2-iminophosphorane (1.0 eq) from procedure 5 and an isocyanate (6.0 eq) in DMAC (dimethylacetamide) (0.75 M in isocyanate) were heated at 70-80° C. for 20-40 h. After cooling to rt, the resin was filtered and washed with DMF, MeOH and CH₂Cl₂ and dried under vacuum. The resin was then treated with 50% TFA in CH₂Cl₂ for 30 min at rt and filtered. The filtrate was collected and dried under vacuum to give the title product as TFA salt.

2-(3′-Methoxyphenylamino)-3-hydroxy-4-quinazolinone TFA salt (75% yield), ¹H NMR (CDCl₃) 8.3 (d, 1H), 7.85 (t, 1H), 7.6 (t, 1H), 7.5 (t, 1H), 7.35 (d, 1H), 7.1-7.2 (m, 3H), 3.9 (s, 3H).

2-(3′-Methoxyphenylamino)-3-hydroxy-7-chloro-4-quinazolinone TFA salt (88% yield), ¹H NMR (CDCl₃) 8.25 (d, 1H), 7.5 (d, 2H), 7.4 (bs, 1H), 7.0-7.2 (m, 3H), 3.9 (s, 3H).

2-(3′-Methoxyphenylamino)-3-hydroxy-6-chloro-4-quinazolinone TFA salt (99% yield), ¹H NMR (CDCl₃) 8.25 (d, 1H), 7.8 (dd, 1H), 7.55 (t, 1H), 7.35 (d, 1H), 7.2 (d, 1H), 7.1 (m, 2H), 3.9 (s, 3H).

2-(3′-Methoxyphenylamino)-3-hydroxy-6-nitro-4-quinazolinone TFA salt (99% yield), ¹H NMR (CDCl₃) 9.1 (d, 1H), 8.6 (dd, 1H), 7.5-7.6 (m, 2H), 7.2 (d, 1H), 7.1 (m, 2H), 3.9 (s, 3H).

2-(3′-Methoxyphenylamino)-3-hydroxy-6-methoxy-4-quinazolinone TFA salt (85% yield), ¹H NMR (CDCl₃) 7.65 (d, 1H), 7.5 (t, 1H), 7.45 (dd, 1H), 7.3 (dd, 1H), 7.15 (dd, 1H), 7.1 (d, 1H), 7.05 (d, 1H), 3.95 (s, 3H), 3.9 (s, 3H).

2-(3′-Nitrophenylamino)-3-hydroxy-4-quinazolinone TFA salt (85% yd), ¹H NMR (CDCl₃) 8.45 (m, 2H), 8.35 (m, 1H), 7.8-7.9 (m, 2H), 7.65 (t, 1H), 7.3 (m, 2H).

2-(3′-Nitrophenylamino)-3-hydroxy-6-methoxy-4-quinazolinone TFA salt (77% yd), ¹H NMR (CDCl₃) 8.45 (m, 2H), 7.8-7.9 (m, 2H), 7.7 (d, 1H), 7.45 (dt, 1H), 7.25-7.3 (dd, 1H), 4.0 (s, 3H).

2-(Cyclohexylamino)-3-hydroxy-4-quinazolinone TFA salt (92% yd), ¹H NMR (CDCl₃) 8.3 (d, 1H), 7.95 (t, 1H), 7.6 (t, 1H), 7.55 (d, 1H), 7.3 (d, 1H) 3.9 (m, 1H), 2.2 (m, 2H, 1.9 (m, 2H), 1.2-1.6 (m, 8H).

2-(Benzylamino)-3-hydroxy-6-chloro-4-quinazolinone TFA salt (99% yd), ¹H NMR (CDCl₃) 8.3 (s, 1H), 7.85 (dd, 1H), 7.3-7.5 (m, 6H), 4.9 (d, 2H).

### General Procedures for Biological Assays

(A) General procedures for 5-LO assay:

5-LO enzyme assays (radio-immuno assays (RIA)) were performed according to literature procedures (ref: (a) Egan, R. W., and Gale, P. H., J. Biol. Chem. 1985, 260:11554-11559; (b) Shimizu, T., Rdamark, O., and Samuelsson, B. Proc. Natl. Acad. Sci. USA, 1984, 81:689-693); (c) Coffey, M. et al., J. Biol. Chem. 1992, 267: 570-576) using commercially available kits or reagents.

### Procedure A

Tissue/cell sources: Rat basophilic leukemia cells (RBL-1);

Substrate: Arachidonic acid (AA) at 14 μM;

Reaction: AA→5-HPETE→5-HETE, incubation at 25° C. for 8 min;

Method: RIA quantitation of 5-HETE, reference compound NGDA (nordihydroquaiaretic acid).

### Procedure B

Tissue/cell sources: Human differentiated HL-60 cells;

Substrate: Arachidonic acid (AA) at 0.4 μM;

Reaction: AA→5-HPETE→5-HETE, incubation at 37° C. for 10 min;

Method: RIA quantitation of 5-HETE, reference compound NGDA (nordihydroquaiaretic acid).

The results are expressed as percent of control activity and as a percent inhibition of control activity obtained in the presence of the tested compounds.

IC₅₀ values (concentration causing a half-maximal inhibition of control activity) and Hill coefficients (nH) are determined by non-linear regression analysis of the concentration-response curves. These parameters are obtained by Hill equation curve fitting.

(B) General procedure for the Histamine H1-receptor (peripheral) Binding Assay:

Histamine H1-receptor (peripheral) binding assays were performed according to the literature procedure (ref: Dini, S., et al, Agents and Actions, 1991, 33:181-184). Aliquots of guinea-pig lung membrane preparations corresponding to 800 μg protein are incubated for 15 min at 22° C. in 500 μl of 50 mM Na₂HPO₄/KH₂PO₄ buffer (pH 7.5) containing 1 nM [³H]pyrilamine and increasing concentrations of the competing drugs. Nonspecific binding is determined in the presence of 100 μM triprolidine. After incubation, the samples are filtered rapidly under vacuum through glass fiber filters (GF/B, Whatman) and rinsed several times with ice-cold 50 mM Na₂HPO₄/KH₂PO₄ using a cell harvester (Brandel). Bound radioactivity is measured with a scintillation counter (LS 6000, Beckman) using a liquid scintillation cocktail (Formula 989, Packard).The reference compound for this assay is pyrilamine.

The results are expressed as percent of control specific binding and as a percent inhibition of control specific binding obtained in the presence of the tested compounds. IC₅₀ values (concentration causing a half-maximal inhibition of control activity) and Hill coefficients (nH) are determined by non-linear regression analysis of the concentration-response curves. These parameters are obtained by Hill equation curve fitting.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A compound having a structure represented by I: 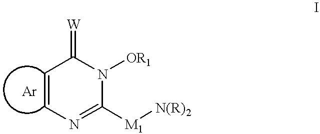
wherein
W represents O or S;
M represents, independently for each occurrence, a methylene group, or two instances of M taken together may form ethene or ethyne;
i represents an integer from 0-6;
R₁ represents H or a group which is cleaved under physiologic conditions;
n represents, independently for each occurrence, an integer from 0-10;
Ar, independently for each occurrence, represents a substituted or unsubstituted aryl or heteroaryl ring; and
N(R)₂ represents: 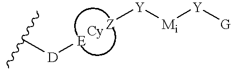
wherein Cy, independently for each occurrence, represents a carbocyclyl, heterocyclyl, aryl, or heteroaryl ring;
D represents NR₈ or is absent;
Z represents C(YR₁), CH, C═, or N;
E represents CH or N;
Y, independently for each occurrence, represents NR₈, O, S, or is absent;
G represents a substituted or unsubstituted heterocyclic ring; aryl ring; diarylmethyl group, optionally additionally substituted with an additional functional group; or polycyclic group, and
R₈ represents, independently for each occurrence, a functional group selected from H, a substituted or unsubstituted alkyl, alkenyl, alkynyl, —(CH₂)ₙaralkyl, —(CH₂)ₙcycloalkyl, —(CH₂)ₙheterocyclyl, —(CH₂)ₙaryl, —(CH₂)ₙCH(aryl)₂, —(CH₂)ₙC(OH)(aryl)₂, —(CH₂)ₙpolycyclyl, and —(CH₂)ₙheteroaryl, or two instances of R₈ taken together may form a 3- to 8-membered cycloalkyl, polycyclyl, or heterocyclyl ring; and
wherein the stereochemical configuration at any stereocenter may be R, S, or a mixture of these configurations.

2. A compound of claim 1, wherein Cy represents a six-membered ring.

3. A compound of claim 1, wherein R₁ represents H.

4. A compound of claim 1, wherein W represents O.

5. A compound of claim 1, wherein M₁ is absent or is lower alkyl.

6. A compound of claim 1, wherein N(R)₂ represents one of: 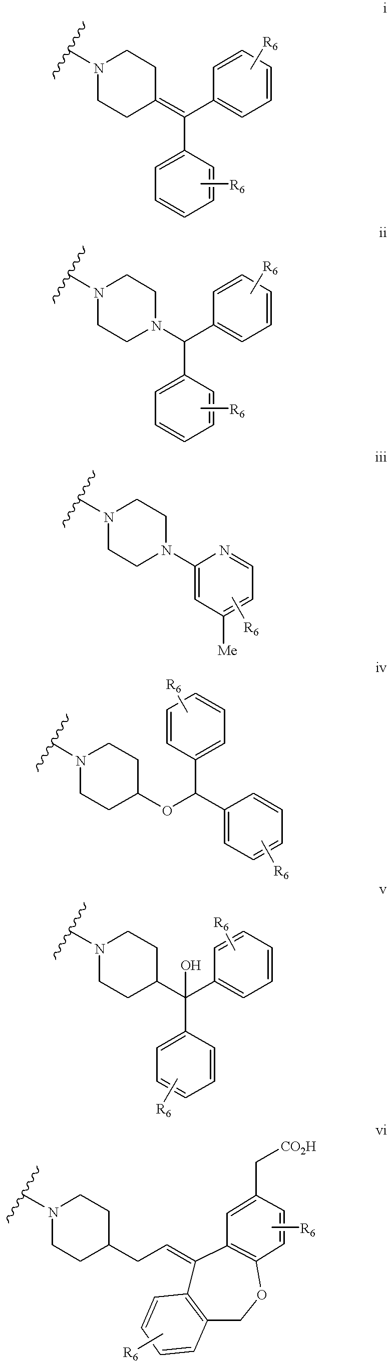
wherein R₆, independently for each occurrence, represents from 0-3 substituents selected from halogen, hydroxyl, lower alkoxy, and lower alkyl.

7. A compound having a structure represented by II: 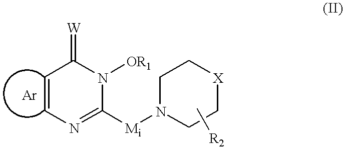
wherein
W represents O or S;
M represents, independently for each occurrence, a methylene group, or two instances of M taken together may form ethene or ethyne;
i represents an integer from 0-6;
R₁ represents H or a group which is cleaved under physiologic conditions;
n represents, independently for each occurrence, an integer from 0-10;
Ar, independently for each occurrence, represents a substituted or unsubstituted aryl or heteroaryl ring; and
X represents NR₈, C═C(R₈)₂, or CH(YR₈);
Y represents NR, O, S, or is absent;
R₂ represents from 0-4 substituents on the ring to which it is attached, and, independently for each occurrence, may represent halogen, a substituted or unsubstituted lower alkyl, lower alkenyl, aryl, or heteroaryl, carbonyl, thiocarbonyl, ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₂ taken together may form a 4- to 8-membered cycloalkyl, heterocyclyl, aryl, or heteroaryl ring;
R₈ represents, independently for each occurrence, a functional group selected from H, a substituted or unsubstituted alkyl, alkenyl, alkynyl, —(CH₂)ₙaralkyl, —(CH₂)ₙcycloalkyl, —(CH₂)ₙheterocyclyl, —(CH₂)ₙaryl, —(CH₂)ₙCH(aryl)₂, —(CH₂)ₙC(OH)(aryl)₂, —(CH₂)ₙpolycyclyl, and —(CH₂)ₙheteroaryl, or two instances of R₈ taken together may form a 3- to 8-membered cycloalkyl, polycyclyl, or heterocyclyl ring; and
p represents an integer from 0-10;
wherein the stereochemical configuration at any stereocenter may be R, S, or a mixture of these configurations.

8. A compound of claim 7, wherein Ar represents a phenyl ring.

9. A compound of claim 7, wherein R₁ represents H.

10. A compound of claim 7, wherein W represents O.

11. A compound of claim 7, wherein Mᵢ is lower alkyl.

12. A compound of claim 7, wherein X includes a group having at least two aryl or heteroaryl rings.

13. A compound of claim 7, wherein X is C═C(R₈)₂ and the two occurrences of R₈ taken together form a ring.

14. A compound having a structure represented by III: 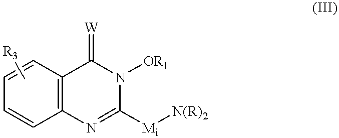
wherein
R₃ represents from 0-4 substituents on the ring to which it is attached, and, independently for each occurrence, may represent halogen, a substituted or unsubstituted lower alkyl, lower alkenyl, aryl, or heteroaryl, carbonyl, thiocarbonyl, ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, O—(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₃ taken together may form a 4- to 8-membered cycloalkyl, heterocyclyl, aryl, and heteroaryl;
W represents O or S;
M represents, independently for each occurrence, a methylene group, or two instances of M taken together may form ethene or ethyne;
i represents an integer from 0-6;
R₁ represents H or a group which is cleaved under physiologic conditions;
n represents, independently for each occurrence, an integer from 0-10;
Ar, independently for each occurrence, represents a substituted or unsubstituted aryl or heteroaryl ring; and
N(R)₂ represents: 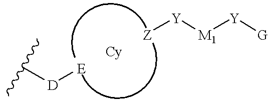
wherein Cy, independently for each occurrence, represents a substituted or unsubstituted carbocyclyl, heterocyclyl, aryl, or heteroaryl ring;
D represents NR₈ or is absent;
Z represents C(YR₁), CH, C═, or N;
E represents CH or N;
Y, independently for each occurrence, represents NR₈, O, S, or is absent;
R₈ represents, independently for each occurrence, a functional group selected from H, substituted or unsubstituted alkyl, alkenyl, alkynyl, —(CH₂)ₙaralkyl, —(CH₂)ₙcycloalkyl, —(CH₂)ₙheterocyclyl, —(CH₂)ₙaryl, —(CH₂)ₙCH(aryl)₂, —(CH₂)ₙC(OH)(aryl)₂, —(CH₂)ₙpolycyclyl, and —(CH₂)ₙheteroaryl, or two instances of R₈ taken together may form a 3- to 8-membered cycloalkyl, polycyclyl, or heterocyclyl ring; and
G represents a heterocyclic ring; a aryl ring; a diarylmethyl group, optionally additionally substituted with an additional functional group; or a polycyclic group,
wherein the stereochemical configuration at any stereocenter may be R, S, or a mixture of these configurations.

15. A compound of claim 14, wherein R₁ represents H.

16. A compound of claim 14, wherein W represents O.

17. A compound of claim 14, wherein Mᵢ is lower alkyl.

18. A compound of claim 14, wherein N(R)₂ represents one of: 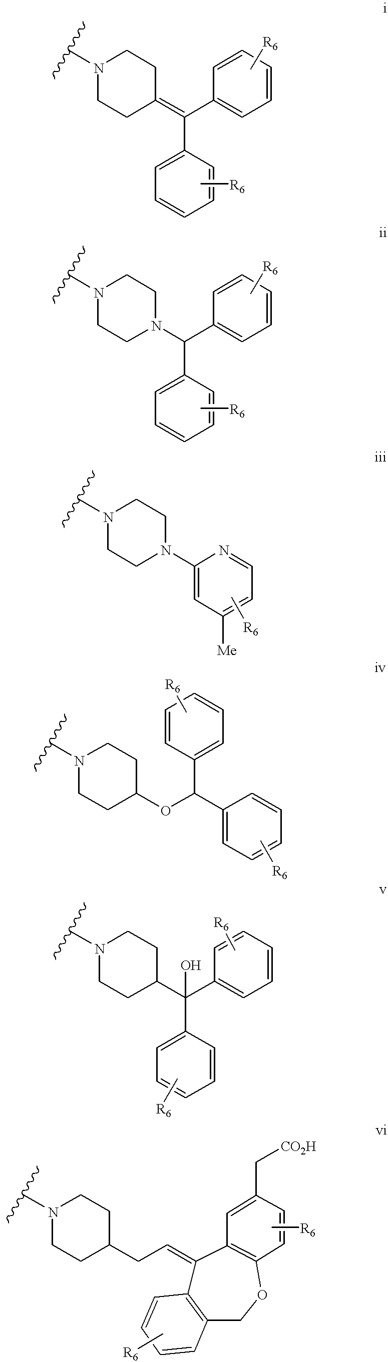
wherein R₆, independently for each occurrence, represents from 0-3 substituents selected from halogen, hydroxyl, lower alkoxy, and lower alkyl.

19. A compound having a structure represented by IV: 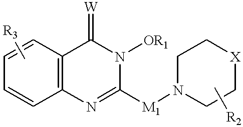
wherein
W represents O or S;
M represents, independently for each occurrence, a methylene group, or two instances of M taken together may form ethene or ethyne;
i represents an integer from 0-6;
R₁ represents H or a group which is cleaved under physiologic conditions;
n represents, independently for each occurrence, an integer from 0-10;
Ar, independently for each occurrence, represents a substituted or unsubstituted aryl or heteroaryl ring; and
X represents NR₈, C═C(R₈)₂, or CH(YR₈);
Y represents NR, O, S, or is absent;
R₂ represents from 0-4 substituents on the ring to which it is attached, and, independently for each occurrence, may represent halogen, a substituted or unsubstituted lower alkyl, lower alkenyl, aryl, or heteroaryl, carbonyl, thiocarbonyl, ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₂ taken together may form a 4- to 8-membered cycloalkyl, heterocyclyl, aryl, or heteroaryl ring;
R₃ represents from 0-4 substituents on the ring to which it is attached, and, independently for each occurrence, may represent halogen, a substituted or unsubstituted lower alkyl, lower alkenyl, aryl, or heteroaryl, carbonyl, thiocarbonyl, ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₂ taken together may form a 4- to 8-membered cycloalkyl, heterocyclyl, aryl, or heteroaryl ring; and
R₈ represents, independently for each occurrence, a functional group selected from H, a substituted or unsubstituted alkyl, alkenyl, alkynyl, —(CH₂)ₙaralkyl, —(CH₂)ₙcycloalkyl, —(CH₂)ₙheterocyclyl, —(CH₂)ₙaryl, —(CH₂)ₙCH(aryl)₂, —(CH₂)ₙC(OH)(aryl)₂, —(CH₂)ₙpolycyclyl, and —(CH₂)ₙheteroaryl, or two instances of R₈ taken together may form a 3- to 8-membered cycloalkyl, polycyclyl, or heterocyclyl ring; and
p represents an integer from 0-10;
wherein the stereochemical configuration at any stereocenter may be R, S, or a mixture of these configurations.

20. A compound of claim 19, wherein R₁ represents H.

21. A compound of claim 19, wherein W represents O.

22. A compound of claim 19, wherein Mᵢ is lower alkyl.

23. A compound of claim 19, wherein X includes a group having at least two aryl or heteroaryl rings, present either independently or fused into a polycyclic group.

24. A compound of claim 19, wherein X is C═C(R₈)₂ and the two occurrences of R₈ taken together form a ring.

25. A compound having a structure represented by I: 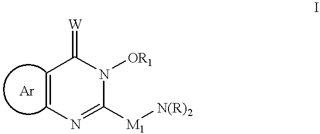
wherein
W represents O or S;
R represents, independently for each occurrence, H or a substituted or unsubstituted alkyl, alkenyl, alkynyl, aralkyl, —(CH₂)ₙcycloalkyl, —(CH₂)ₙheterocyclyl, —(CH₂)ₙaryl, or —(CH₂)ₙheteroaryl, or two instances of R taken together with the nitrogen to which they are attached may form a ring of between 3 and 8 atoms which may include 1 or 2 additional heteroatoms, and may be substituted with 1 or 2 substituents selected from alkyl, alkenyl, alkynyl, aralkyl, —(CH₂)ₙcycloalkyl, —(CH₂)ₙheterocyclyl, —(CH₂)ₙaryl, and —(CH₂)ₙheteroaryl;
M represents, independently for each occurrence, a methylene group or two instances of M taken together may form ethene or ethyne;
i represents an integer from 0-6;
R₁ represents H or a group which is cleaved under physiologic conditions;
n represents, independently for each occurrence, an integer from 0-10;
Ar represents a substituted or unsubstituted aryl or heteroaryl ring; and
the stereochemical configuration at any stereocenter may be R, S, or a mixture of these configurations.

26. A pharmaceutical composition, comprising a pharmaceutically acceptable excipient and a compound of claim 1, 7, 14, 19, or 25.

27. A method for inhibiting activity of 5-LO and Hi in a patient, comprising administering to the patient a therapeutically effective amount of a compound of claim 1, 7, 14, 19, or 25.

28. A method for treating asthma in a patient, comprising administering to the patient a therapeutically effective amount of a compound of claim 1, 7, 14, 19, or 25.

29. A method for treating an allergic condition in a patient, comprising administering to the patient a therapeutically effective amount of a compound of claim 1, 7, 14, 19, or 25.

30. The method of claim 29, wherein the allergic condition is selected from dermatitis, urticaria, allergic rhinitis, and allergic conjunctivitis.

31. A method for preparing N-hydroxyquinazolinones or derivatives thereof, comprising reacting an anthranilic acid derivative having the formula V: 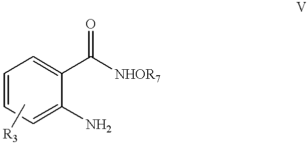
with an activated phosphine compound and an isocyanate or isothiocyanate having the formula R-NCX to generate an N-hydroxyquinazolinone compound having the formula VI: 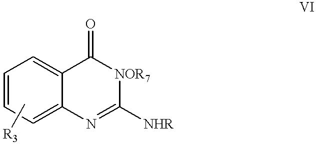
wherein X represents S or O;
R represents alkyl, alkenyl, alkynyl, aralkyl, —(CH₂)ₙcycloalkyl, —(CH₂)ₙheterocyclyl, —(CH₂)ₙaryl, or —(CH₂)ₙheteroaryl, or —(CH₂)ₙpolycyclyl;
R₃ represents from 0-4 substituents on the ring to which it is attached, and, independently for each occurrence, may represent halogen, lower alkyl, lower alkenyl, aryl, heteroaryl, carbonyl, thiocarbonyl, ketone, aldehyde, amino, acylamino, amido, amidino, hydroxyl, alkoxy, cyano, nitro, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR₈, —(CH₂)ₚSH, —(CH₂)ₚS-lower alkyl, —(CH₂)ₚS-lower alkenyl, —S(CH₂)ₙR₈, —(CH₂)ₚN(R₈)₂, —(CH₂)ₚNR₈-lower alkyl, —(CH₂)ₚNR₈-lower alkenyl, —NR₈(CH₂)ₙR₈, or protected forms of the above, or any two instances of R₂ taken together may form a 4- to 8-membered cycloalkyl, heterocyclyl, aryl, or heteroaryl ring; and
R₇ represents acyl, sulfonyl, sulfinyl, alkyl, alkenyl, alkynyl, aralkyl, —(CH₂)ₙcycloalkyl, —(CH₂)ₙheterocyclyl, —(CH₂)ₙaryl, or —(CH₂)ₙheteroaryl, or —(CH₂)ₙpolycyclyl.

32. The method of claim 31, wherein the activated phosphine compound is the product of a phosphine and an azodicarboxylate ester.

33. The method of claim 31, wherein the activated phosphine compound is the product of a halogen and a phosphine.

34. The method of claim 31, wherein the activated phosphine compound is a phosphorous (V) compound capable of reacting with an aniline to form an iminophosphorane.

35. The method of claim 31, wherein the activated phosphine compound has the formula Ar₃PX₂, wherein X, independently for each occurrence represents a leaving group, and Ar represents a substituted or unsubstituted aryl or heteroaryl group.
